(19) United States
(12) Patent Application Publication
(10) Pub. No.: US 2026/0294589 A1
(43) Pub. Date: Oct. 1, 2026

(54) METHODS AND SYSTEMS FOR ORTHODONTIC TREATMENT PLANNING

(71) Applicant: Align Technology, Inc., San Jose, CA (US)

(72) Inventors: Valerii IVANOV, Madrid (ES); Anna LUKINYKH, Madrid (ES); Arshak KOCHARYAN, Yerevan (AM); Artem TANSAN, Moscow (RU); Viktoriia KONSTANTINOVA, Yerevan (AM); Anastasiia KORABLEVA, Frankfurt am Main (DE); Nelli KUDRYAVTSEVA, Raleigh, NC (US); Alexander PORYVKIN, Moscow (RU); Anton RUDIKOV, Yerevan (AM); Aleksei SEKACHEV, Moscow (RU); Ivan MURANOV, Morrisville, NC (US)

(21) Appl. No.: 19/578,810

(22) Filed: Mar. 25, 2026

Related U.S. Application Data
(60) Provisional application No. 63/777,641, filed on Mar. 25, 2025.

(51) Int. Cl. A61C 7/00; A61C 7/08; A61C 7/16

(52) U.S. Cl. CPC A61C 7/002; A61C 7/08; A61C 7/16

(57) ABSTRACT

Methods and apparatuses for displaying modifying and/or updating a patient's dental treatment plan are disclosed. As a dental treatment plan is revised or updated, a collision or interference between aligner attachments is detected and the user is alerted. In some examples, an occlusal feature can be added to the patient's dental treatment plan through interaction with a user interface. In some other examples, one or more aligner attachment can be identified and removed from a dental treatment plan while still providing a desired amount of tooth movement.

### CLAIM OF PRIORITY

This patent application claims priority to U.S. provisional patent application No. 63/777,641, titled “METHODS AND SYSTEMS FOR ORTHODONTIC TREATMENT PLANNING,” and filed on Mar. 25, 2025, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The systems and methods described herein relate generally to analysis of dental images, and more particularly to the determination and viewing of alternate views of three-dimensional dental models corresponding to a dental treatment plan.

### BACKGROUND

Orthodontic and dental treatments using a series of patient-removable appliances (e.g., “aligners”) are very useful for treating a variety of patients. Treatment planning is typically performed in conjunction with the dental professional (e.g., dentist, orthodontist, dental technician, etc.), by manipulating a model of the patient's teeth from an initial configuration (initial tooth positions) to a final configuration (final tooth positions) and then dividing the treatment into a number of intermediate stages (steps). These steps may correspond to individual appliances that may be worn sequentially, with or without additional interventions (e.g., interproximal reductions, extractions, etc.). Once the treatment plan is finalized, the series of aligners may be manufactured corresponding to the treatment plan.

The treatment plan may begin with a dental scan of the patient's dentition. The dental scan can be the basis of the treatment plan and a three-dimensional (3D) model of the patient's teeth may be generated as part of the treatment plan. In some examples, the aligners may be manufactured using information from the three-dimensional model.

In some instances, a treatment plan may be revised after treatment of the patient has begun. For example, after a few months of treatment, a patient's dentition may be rescanned to check for the effectiveness of the dental treatment thus far. The updated dental scan may show tooth movement deviations from the treatment plan. The deviations may be significant enough to warrant a revision (or update) of the dental treatment plan.

Conventionally, revising the treatment plan can be a time consuming and/or complex task. The dental professional (dentist, orthodontist, or other clinician) needs to consider procedures that were included in the previous treatment plan. In addition, some treatment plan revisions may benefit from a shorter revise and review cycle.

### SUMMARY OF THE DISCLOSURE

Described herein are apparatuses (e.g., systems, devices, etc., including software, hardware and/or firmware) and methods for generating, modifying and/or updating a patient's dental treatment plan. In particular, various apparatuses and methods enable a user (dentist, orthodontist, or other clinician) to interact with a three-dimensional (3D) digital model of a patient's dentition. In some examples, the user can update the dental treatment plan based on an updated dental scan. A collision or interference between existing aligner attachment (from an earlier dental treatment plan) and a new aligner attachment (from a subsequent dental treatment plan) is detected and the user is alerted through a user interface. The user interface can also enable the user to resolve the attachment conflict.

In another example, the various apparatuses and methods enable the user to add occlusal features to the patient's dental treatment plan. The user can select occlusal surfaces to receive any feasible occlusal feature. After the occlusal feature is added to the dental treatment plan, the occlusal feature can be added to one or more related dental aligners.

In another example, the various apparatuses and methods enable the user to identify and remove aligner attachments from the patient's dental treatment plan. For example, the user can select particular tooth and determine whether the tooth displacement for selected tooth is feasible without an aligner attachment. The feasible, the dental treatment plan is revised.

In some examples, various views of the patient's 3D digital model may be generated and displayed to the user through an appropriate user interface. The user can interact with the 3D digital model as well as other user interface features (menus, buttons, and the like) to update the patient's dental treatment plan.

Any of the systems described herein may be used to design a dental treatment plan and can include a user interface configured to display graphical data and receive user input, one or more processors, and a memory coupled to the one or more processors, the memory configured to store computer-program instructions that, when executed by the one or more processors cause the system to implement a method comprising displaying, on a user interface, a three-dimensional (3D) digital model of a dentition based on a dental treatment plan, wherein the 3D digital model includes a surface of at least one tooth with a first attachment, receiving, through the user interface, a user input to locate a second attachment on the surface of the at least one tooth, determining, by the one or more processors, an interference between the first attachment and the second attachment, generating an updated dental treatment plan based on the interference between the first attachment and the second attachment, and displaying an updated 3D digital model based on the updated dental treatment plan.

In any of the systems described herein, the 3D digital model may be based on a dental scan completed after treatment according to the dental treatment plan has begun. In any of the systems described herein, the updated dental treatment plan can remove the first attachment. In place of the second attachment.

Any of the systems described herein can also display, on the user interface, the interference between the first attachment and the second attachment. In this manner, interferences between attachments can be displayed and/or resolved.

In any of the systems described herein can include, generating the updated dental treatment plan can include sending the user input to a remote computing device configured to generate the updated dental treatment plan based on the dental treatment plan and returning the updated dental treatment plan to the user interface.

In any of the systems described herein can include sending the user input to a designer to generate the updated dental treatment plan based on the dental treatment plan and returning the updated dental treatment plan to the user interface.

Any of the systems described herein can include displaying, on the user interface, the interference between the first attachment and the second attachment, based on determining the interference between the first attachment and the second attachment. Furthermore, the systems can include displaying, on the user interface, the interference between the first attachment and the second attachment, based on determining the interference between the first attachment and the second attachment.

A non-transitory computer-readable storage medium can include instructions that, when executed by one or more processors of a device, cause the device to perform operations comprising displaying, on a user interface, a three-dimensional (3D) digital model of a dentition based on a dental treatment plan, wherein the 3D digital model includes a surface of at least one tooth with a first attachment, receiving, through the user interface, a user input to locate a second attachment on the surface of the at least one tooth, determining, by the one or more processors, an interference between the first attachment and the second attachment, generating an updated dental treatment plan based on the interference between the first attachment and the second attachment, and displaying an updated 3D digital model based on the updated dental treatment plan.

Any of the systems described herein can be used for designing a dental treatment plan and can include a user interface configured to display graphical data and receive user input, one or more processors, a memory coupled to the one or more processors, the memory configured to store computer-program instructions that, when executed by the one or more processors cause the system to implement a method comprising displaying, on a user interface, a three-dimensional (3D) digital model of a dentition based on a patient's dental treatment plan, receiving, through the user interface, a user input selecting a location for an occlusal feature on a tooth of the 3D digital model, determining, by the one or more processors, whether the occlusal feature can be implemented at the selected location, generating an updated dental treatment plan based at the selected location of the occlusal feature, and displaying an updated 3D digital model based on the updated dental treatment plan.

In any of the systems described herein, the occlusal feature can include a bite ramp. In some other systems, the occlusal feature can include a disocclusion feature. In any of the systems described herein, the occlusal feature comprises a bite ramp shaped to disocclude a first jaw from a second jaw. In any of the systems described herein the occlusal feature can be integral to a dental appliance manufactured in accordance with the updated dental treatment plan.

In any of the systems described herein can include displaying, on the user interface, an indication to a user whether the occlusal feature can be implemented at the selected location. Any of the systems described herein can include receiving through the user interface the user input to remove the occlusal feature.

Any of the systems described herein can also include sending the user input selecting the location for an occlusal feature to a remote computing device configured to generate the updated dental treatment plan and returning the updated dental treatment plan that includes the occlusal feature to the user interface.

Any of the systems described herein can also include receiving, through the user interface, a modification of a previously selected location of the occlusal feature. Other systems described herein can include determining whether the occlusal feature can be implemented at the selected location includes determining whether the occlusal feature can be implemented with respect to a tooth associated with the selected location.

In any of the systems described herein, generating the updated dental treatment plan can include sending the user input to a remote computing device configured to generate the updated dental treatment plan based on the user input and returning the updated dental treatment plan to the user interface.

A non-transitory computer-readable storage medium comprising instruction that, when executed by one or more processors of a device, cause the device to perform operations can include displaying, on a user interface, a three-dimensional (3D) digital model of a dentition based on a patient's dental treatment plan, receiving, through the user interface, a user input selecting a location for an occlusal feature on a tooth of the 3D digital model, determining, by the one or more processors, whether the occlusal feature can be implemented at the selected location, generating an updated dental treatment plan based at the selected location of the occlusal feature, and displaying an updated 3D digital model based on the updated dental treatment plan.

Any of the systems described herein for designing a dental treatment plan can include a user interface configured to display graphical data and receive user input, one or more processors, and a memory coupled to the one or more processors, the memory configured to store computer-program instructions that, when executed by the one or more processors cause the system to implement a method including displaying, on the user interface, a three-dimensional (3D) digital model of a dentition based on a patient's dental treatment plan including one or more locations for one or more aligner attachments that, when engaged with corresponding one or more wells on dental appliances, cause the dental appliances to impart one or more orthodontic forces to the dentition, receiving, through the user interface, a user input selecting a first location of a specified aligner attachment of the one or more aligner attachments, wherein the specified aligner attachment is associated with a selected tooth on the 3D digital model, determining, by the one or more processors, whether the specified aligner attachment can be removed from the selected tooth based on tooth movements of the selected tooth described in the patient's dental treatment plan, determining, a displacement between a current dental appliance and a subsequent dental appliance, wherein the displacement implements the tooth movements of the selected tooth described in the patient's dental treatment plan, and displaying an updated 3D digital model based on the determined displacements between the current dental appliance and the subsequent dental appliance.

Any of the systems described herein can include indicating, on the user interface, that the specified attached cannot be removed from the patient's dental treatment plan based on whether the specified aligner attachment can be removed from the selected tooth.

In any of the systems described herein, determining whether the specified aligner attachment can be removed is based at least in part on an amount of intrusion of the selected tooth described in the dental treatment plan.

Any of the systems described herein can include receiving, through the user interface, an indication to continue to use the specified aligner attachment with respect to the dental treatment plan.

Any of the systems described herein can include generating an updated dental treatment plan based on the updated 3D digital model. In some examples, generating the updated dental treatment plan can include sending the user input to a remote computing device configured to generate the updated dental treatment plan based on the dental treatment plan and returning the updated dental treatment plan to the user interface. In some examples, generating the updated dental treatment plan can include sending the user input to a designer to generate the updated dental treatment plan based on the dental treatment plan and returning the updated dental treatment plan to the user interface.

Any of the non-transitory computer-readable storage mediums described herein can include instructions that, when executed by one or more processors of a device, cause the device to display, on the user interface, a three-dimensional (3D) digital model of a dentition based on a patient's dental treatment plan including one or more locations for one or more aligner attachments that, when engaged with corresponding one or more wells on dental appliances, cause the dental appliances to impart one or more orthodontic forces to the dentition, receive, through the user interface, a user input selecting a first location of a specified aligner attachment of the one or more aligner attachments, wherein the specified aligner attachment is associated with a selected tooth on the 3D digital model, determine, by the one or more processors, whether the specified aligner attachment can be removed from the selected tooth based on tooth movements of the selected tooth described in the patient's dental treatment plan, determine, a displacement between a current dental appliance and a subsequent dental appliance, wherein the displacement implements the tooth movements of the selected tooth described in the patient's dental treatment plan, and display an updated 3D digital model based on the determined displacements between the current dental appliance and the subsequent dental appliance.

For example, described herein are computer-implemented systems for updating a dental treatment plan. These systems may include: a user interface configured to display a three dimensional (3D) digital model of a patient's dentition and to receive user input; one or more processors; and a memory storing instructions that, when executed by the one or more processors, cause the system to perform operations comprising: rendering, on the user interface, a 3D digital model including at least one tooth having a first attachment represented as a 3D geometric mesh integrated with the tooth surface; receiving, through the user interface, a user specified placement location for a second attachment on a surface of the same tooth; determining an attachment interference by: computing a geometric intersection between a volumetric region of the first attachment and a volumetric region of the second attachment using a surface mesh collision detection algorithm; and identifying an overlap exceeding a predefined spatial threshold; generating an updated dental treatment plan by applying one or more attachment resolution rules that include at least one of: automatically removing the first attachment if a clinical rule permits substitution with the second attachment, automatically removing the second attachment when the first attachment must remain, generating a user resolvable conflict state for subsequent review by a remote treatment planning engine; and displaying, on the user interface, an updated 3D digital model that reflects the attachment resolution outcome, including a visual indication of any detected interference.

Any of these apparatuses (systems, devices, etc.) and methods may be configured for placement of one or more occlusal feature (e.g., a bite ramp). For example, described herein are computer implemented systems for inserting an occlusal feature into a dental treatment plan, the system comprising: a user interface configured to display graphical data and receive user input; one or more processors; and a memory storing instructions that, when executed by the one or more processors, cause the system to perform operations comprising: displaying, on the user interface, a 3D digital model of a patient's dentition associated with a dental treatment plan, including occlusal surfaces of anterior teeth; receiving a user selected placement location for an occlusal feature on a tooth surface of the 3D model; determining whether the occlusal feature can be placed at the user selected location by applying an occlusal feature feasibility algorithm comprising: identifying whether the target tooth belongs to a permitted tooth class for the requested feature; evaluating geometric constraints on the 3D tooth surface; and determining whether placement satisfies rule based occlusal interaction criteria stored in a treatment planning constraint set; generating an updated dental treatment plan that incorporates the occlusal feature when the feasibility determination is positive; and rendering, on the user interface, an updated 3D digital model including a representation of the occlusal feature and an indication of placement feasibility.

Any of these methods and apparatuses may also be configured for attachment-free aligner activation. For example, a computer-implemented system for determining whether an aligner attachment can be removed from a dental treatment plan may include: a user interface configured to display graphical data and receive user input; one or more processors; and a memory storing instructions that, when executed by the one or more processors, cause the system to perform operations comprising: displaying, on the user interface, a 3D digital model of a patient's dentition that identifies locations of aligner attachments associated with a multi stage dental treatment plan; receiving, from the user interface, a selection of a first tooth having a corresponding specified attachment; determining whether the specified attachment can be removed by applying a tooth movement feasibility algorithm comprising: obtaining planned tooth movement parameters for the selected tooth, including planned intrusion, mesial distal translation, and root center displacement; comparing the planned movements to predefined attachment free thresholds that include at least an intrusion threshold of 0.5 mm; and determining whether aligner only forces can accomplish the planned movement without the specified attachment; generating a modified dental treatment plan that removes the specified attachment when the feasibility determination is positive and retains the attachment when negative; determining, for each applicable treatment stage, a displacement between a current aligner and a subsequent aligner based on the modified dental treatment plan; and displaying, on the user interface, an updated 3D digital model representing tooth positions corresponding to the modified dental treatment plan.

For example, described herein are systems and methods for real-time evaluation and modification of dental treatment plans using a three-dimensional (3D) digital model of a patient's dentition. A system may include a user interface that displays a 3D representation of the dentition and receives user-initiated modification requests involving attachment placement, attachment removal, occlusal-feature placement, or attachment-free aligner-activation features. The system may apply one or more feasibility-detection algorithms that incorporate rule-based and geometric constraints, such as precomputed tooth-surface constraint maps, tooth-type eligibility rules, color-coded placement indicators, and blocking logic preventing impermissible requests from being forwarded to a treatment-planning engine.

Any of these apparatuses and methods may include a dynamic treatment-plan modification process allows users to place virtual modifications on the 3D model. The system may generate placeholder objects representing such modifications, where the placeholder objects can be interactively selected, repositioned, or deleted. Before a modification is accepted, the system may evaluate potential conflicts with geometric, clinical, or white-attachment-based restrictions and may attach metadata describing each accepted modification. If a modification is later found incompatible during automated treatment-plan recalculation, the system may automatically remove the corresponding placeholder object.

Also described herein are apparatuses and methods that perform dual-stage collision detection and resolution for aligner attachments. A 3D rendering engine may visualize both existing and proposed attachments, while a first-stage local collision-detection module performs preliminary analyses such as mesh-intersection testing. A second-stage remote collision-resolution engine may resolve attachment conflicts during treatment-plan recalculation by determining whether an existing attachment should be retained, a new attachment substituted, or both presented as user-resolvable alternatives. The system may visually indicate the outcome of collision assessment, including color-coded representations of the attachments involved.

Any of these apparatuses and methods may include computer-readable media storing instructions for determining whether an attachment can be removed based on tooth-movement requirements. Such implementations may analyze tooth-movement parameters, including planned intrusion levels, to determine whether aligner-only forces can accomplish the planned movement. Updated treatment plans may incorporate recalculated aligner sequences and may visually highlight teeth for which attachment removal is permissible. Removal decisions may be transmitted to downstream aligner-fabrication systems.

Also described herein are apparatuses and methods for automated placement of occlusal features such as bite ramps or disocclusion features. A user interface may allow placement of such features directly on the 3D model. A tooth-classification engine may determine whether the selected tooth is eligible to receive the occlusal feature. A geometric-analysis module may evaluate the suitability of the selected region using curvature, inclination, or bonding-area criteria. An occlusion-interaction evaluator may simulate closing bites to detect undesirable inter-arch contacts, while a placement-decision engine may reject occlusal-feature placement when planned tooth movements are incompatible with the feature.

Also described herein are apparatuses and methods that support multiple simultaneous treatment-plan modifications. The device may process requests involving attachments, occlusal features, and attachment-free aligner-activation through a combination of rule-based evaluations and geometric analysis. The system may apply feasible features to the 3D model, reject infeasible features by disabling their placement options, store feasible features as interactive placeholder objects, and transmit finalized treatment plans to aligner-fabrication systems.

Any of these apparatuses and methods may address consistency issues related to white attachments appearing in secondary treatment plans following rescans. White attachments may be mapped to restricted placement regions, and attempting to place a new attachment within such regions may trigger placement warnings, visual indicators, or workflow-based resolution options. As described herein, a live update process may automatically remove new attachments placed within restricted regions, while a submit process may forward the conflict to a CAD designer for resolution.

Also described herein are apparatuses and methods that provide a system employing multi-criteria frontend evaluation to prevent submission of impermissible modifications to backend treatment-planning engines. This frontend evaluation may apply clinical rules, geometric analyses, spacing thresholds, coordinate-mapping constraints, curvature or slope analysis, prior-attachment occupancy checks, and occlusal-interference prediction. Submission controls may be disabled when modifications fail evaluation, and permissible modifications may be stored in a staging buffer before being forwarded for backend processing.

Collectively, these apparatuses (e.g., systems, devices, media, etc.) and methods described herein may enable efficient, intuitive, and clinically accurate modification of dental treatment plans through real-time analysis, automated feasibility evaluation, collision detection, occlusal-feature assessment, attachment-removal logic, and multi-criteria validation mechanisms.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a diagram illustrating one variation of a dental computing environment.

FIG. 2 is a block diagram of one example of interactive user interface modules.

FIG. 3 is a block diagram of one example of treatment planning modification modules.

FIGS. 4A-4F show a user interface for use in the detection of interfering or colliding attachments.

FIG. 5 is a flowchart showing an example method for designing a dental treatment plan.

FIG. 6 is a flowchart showing an example method for designing a dental treatment plan.

FIGS. 7A-7K show a user interface for use in the addition and management of occlusal features into a dental treatment plan.

FIG. 8 is a flowchart showing an example method for designing a dental treatment plan.

FIG. 9 is a flowchart showing an example method for designing a dental treatment plan.

FIGS. 10A-10K show a user interface for use in the identification of attachments which can be removed from teeth with respect to a patient's treatment plan.

FIG. 11 is a flowchart showing an example method for designing a dental treatment plan.

FIG. 12 is a flowchart showing an example method for designing a dental treatment plan.

FIG. 13 shows a block diagram of a device that may be one example a device configured to perform one or more operations associated with the system of FIG. 1.

### DETAILED DESCRIPTION

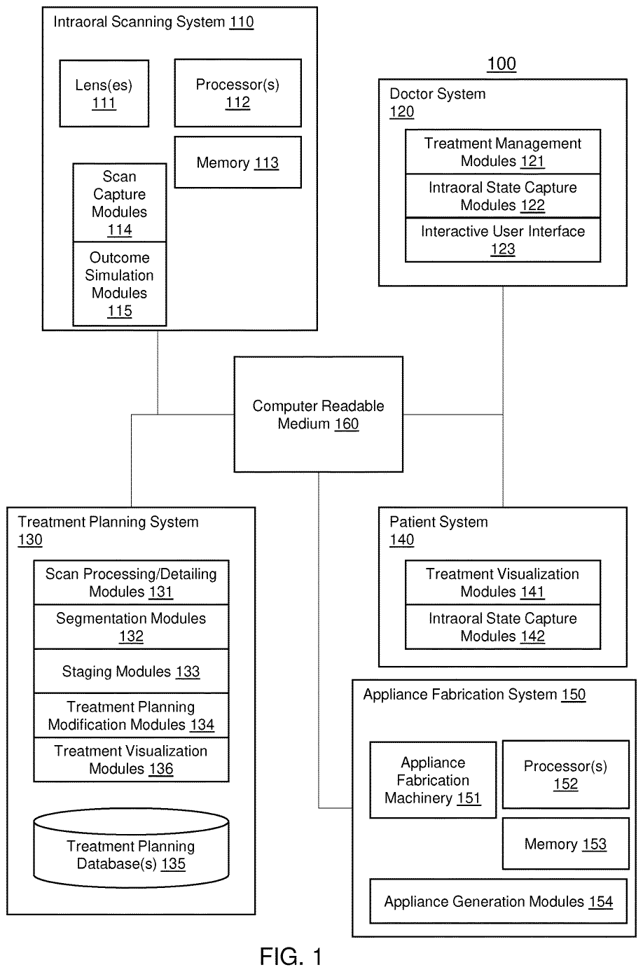

In general, these methods and apparatuses may be used at one or more parts of a dental computing environment, including as part of an intraoral scanning system, doctor system, treatment planning system, patient system, and/or fabrication system. In particular, these methods and apparatuses may be used as part of a treatment planning system, for example, to determine an accurate (in some cases initial) location of a patient's teeth. The initial location may be used to determine a final location for one or more of the patient's teeth based on a dental treatment plan. The dental treatment plan may be optimized (modified) based on updated dental scans. For example, FIG. 1 is a diagram illustrating one variation of a dental computing environment 100 that may generate one or more orthodontic or dental treatment plans specific to a patient and fabricate dental appliances that may accomplish the treatment plan to treat a patient under the direction of a dental professional. The example dental computing environment 100 shown in FIG. 1 includes an intraoral scanning system 110, a doctor system 120, a treatment planning system 130, a patient system 140, an appliance fabrication system 150, and computer-readable medium 160. In some variations the dental computing environment (sometimes referred to as a dental computing system) 100 may include just one or a subset of these systems (which may also be referred to as sub-systems of the overall system 100). Further, one or more of these systems may be combined or integrated with one or more of the other systems (sub-systems), such as, e.g., the treatment planning system 130 and the doctor system 120 may be part of a remote server accessible by a doctor interface. The computer-readable medium 160 may divided between all or some of the systems (subsystems); for example, the treatment planning system 130 and the appliance fabrication system 150 may be part of the same sub-system and may be on a computer-readable medium 160. Further, each of these systems may be further divided into sub-systems or components that may be physically distributed (e.g., between local and remote processors, etc.) or may be integrated.

The intraoral scanning system 110 may include an intraoral scanner as well as one or more processors for processing images. For example, the intraoral scanning system 110 can include lens(es) 111, processor(s) 112, a memory 113, scan capture modules 114, and outcome simulation modules 115. In general, the intraoral scanning system 110 can capture one or more images of a patient's dentition. Use of the intraoral scanning system 110 may be in a clinical setting (doctor's office or the like) or in a patient-selected setting (the patient's home, for example). In some cases, operations of the intraoral scanning system 110 may be performed by an intraoral scanner, dental camera, cell phone or any other feasible device.

The lens(es) 111 include one or more lenses and/or optical sensors to capture reflected light, particularly from a patient's dentition. The scan capture modules 114 can include instructions (such as non-transitory computer-readable instructions) that may be stored in the memory 113 and executed by the processor(s) 112 to control the capture of any number of images of the patient's dentition.

In some examples, the methods and apparatuses described herein for generating a three-dimensional (3D) digital model including one or more teeth may be part of, or accessible by, the intraoral scanning system 110, computer-readable medium 160 and/or treatment planning system 130.

For example, the outcome simulation modules 115, which may be part of the intraoral scanning system 110, can include instructions that simulate final tooth positions (e.g., a predicted configuration of teeth) based on a dental treatment plan. In some cases, the outcome simulation modules 115 can include instructions that simulate tooth positions using images or other scan data from a dental scan.

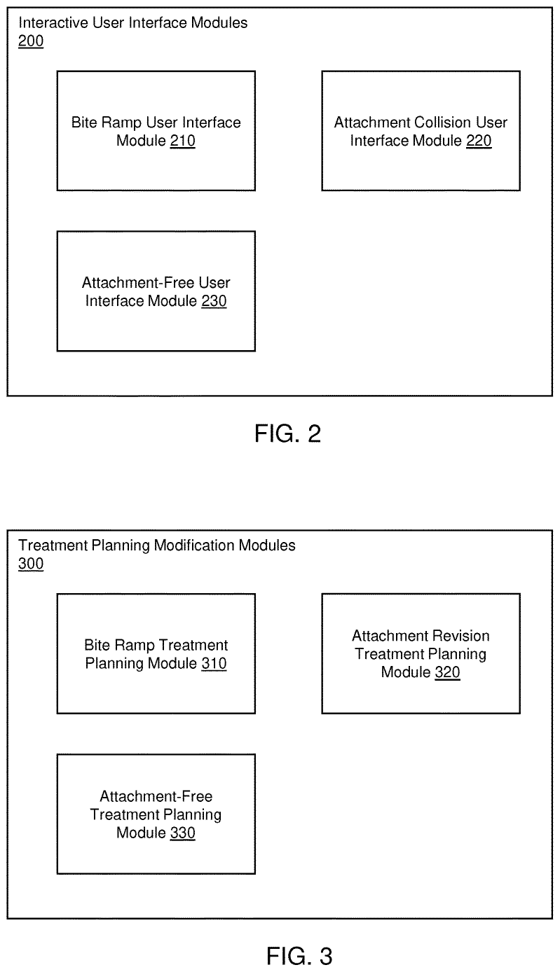

Any of the component systems or sub-systems of the dental computing environment 100 may access or use the patient's dental information including scan data, 3D digital models, and/or dental treatment plans generated by the methods and apparatuses described herein. For example, the doctor system 120 may include treatment management modules 121 and intraoral state capture modules 122 that may access or use patient scan data and/or 3D digital models. The doctor system 120 may provide a “doctor facing” interface to the computing environment 100. The treatment management modules 121 can perform any operations that enable a doctor or other clinician to manage the treatment of any patient. In some examples, the treatment management modules 121 may provide a visualization and/or simulation of the patient's dentition with respect to a dental treatment plan. For example, the doctor system 120 may include a user interface for the doctor that allows the doctor to manipulate and view a patient's 3D digital model, including a 3D digital model corresponding to a final position of teeth in accordance with the dental treatment plan. More details regarding the generation of a view of the patient's 3D dental model is described herein with respect to FIG. 2.

The intraoral state capture modules 122 can provide images of the patient's dentition to a clinician through the doctor system 120. The images may be captured through the intraoral scanning system 110 and may also include images of a simulation of tooth movement based on a dental treatment plan.

In some examples, the treatment management modules 121 can enable the doctor to modify or revise a dental treatment plan. The doctor system 120 may include one or more processors configured to execute any feasible non-transitory computer-readable instructions to perform any feasible operations described herein.

In some examples, the doctor system 120 can include an interactive user interface 123. The interactive user interface 123 enables a clinician or other dental professional to monitor a patient's progress with respect to a patient's dental treatment plan. The interactive user interface 123 may include a display to show graphic images and implement a graphical user interface. The interactive user interface 123 can also enable a user to modify a dental treatment plan and visualize predicted outcomes of the dental treatment plan changes. For example, the interactive user interface 123 can include one or more of a keyboard, a pointing device, a tablet, touchscreen, or the like to receive user input, particularly input which may be used to modify a dental treatment plan. In some examples, the interactive user interface may be used insert bite ramps, detect attachment collisions and/or revise a dental treatment plan to avoid the use of attachments. The interactive user interface 123 is described in more detail with respect to FIG. 2.

Alternatively or additionally, the treatment planning system 130 may include any of the methods and apparatuses described herein, and/or may determine a mapping between dental scans of a patient that are displaced in time. The treatment planning system 130 may include scan processing/detailing modules 131, segmentation modules 132, staging modules 133, treatment planning modification modules 134, treatment visualization modules 136, and treatment planning database(s) 135. In general, the treatment planning system 130 can determine a dental treatment plan for any feasible patient. The scan processing/detailing modules 131 can receive or obtain dental scans (such as scans from the intraoral scanning system 110) and can process the scans to “clean” them by removing scan errors and, in some cases, enhancing details of the scanned image.

The treatment planning system 130 may include a segmentation system that segments a model into separate components. For example, the treatment planning system 130 may include a segmentation modules 132 that can segment a digital model (such as a 3D digital dental model) into separate parts including separate teeth, gums, jaw bones, and the like. In some cases, the digital models may be based on scan data from the scan processing/detailing modules 131.

The staging modules 133 may determine different stages of a dental treatment plan. Each stage may correspond to a different dental aligner. In some examples, the staging modules 133 may also determine the final position (also referred to as target position) of the patient's teeth, in accordance with a dental treatment plan. Thus, the staging modules 133 can determine some or all of a patient's orthodontic or dental treatment plan. In some examples, the staging modules 133 can simulate movement of a patient's teeth in accordance with the different stages of the patient's dental treatment plan.

The treatment planning modification modules 134 can change or modify one or more aspects of a patient's dental treatment plan. In some examples, the treatment planning modification modules 134 can receive user input (such as through the interactive user interface 123) to change or modify a dental treatment plan and provide an analysis of the dental treatment plan changes to a clinician. In some examples, the treatment planning modification modules 134 may be used to adjust or apply bite ramps, revise attachment placement, and/or revise treatment plans to avoid the use of attachments. Treatment planning modification modules 134 are described in more detail with respect to FIG. 3. The dental treatment plans may be stored in the treatment planning database(s) 135. Although not shown here, the treatment planning system 130 can include one or more processors configured to execute any feasible non-transitory computer-readable instructions to perform any feasible operations described herein.

The treatment visualization modules 136 can perform any operations that enable a doctor or other clinician to visualize the dental treatment plan of any patient. In some examples, the treatment visualization modules 136 may provide a visualization and/or simulation of the patient's dentition with respect to a proposed dental treatment plan. For example, the treatment planning system 130 may include a user interface that allows the doctor to manipulate and/or view a patient's 3D digital model, including a 3D digital model corresponding to a final position of teeth in accordance with the dental treatment plan. More details regarding the generation of a view of the patient's 3D digital model is described herein with respect to FIG. 2.

The patient system 140 can include treatment visualization modules 141 and intraoral state capture modules 142. In general, the patient system 140 can provide a “patient facing” interface to the computing environment 100. The treatment visualization modules 141 can enable the patient to visualize how a dental treatment plan has progressed and also visualize a predicted outcome (e.g., a final position of teeth).

In some examples, the patient system 140 can capture dentition scans for the treatment visualization modules 141 through the intraoral state capture modules 142. The intraoral state capture modules 142 can enable a patient to capture his or her own dentition through the intraoral scanning system 110. Although not shown here, the patient system 140 can include one or more processors configured to execute any feasible non-transitory computer-readable instructions to perform any feasible operations described herein.

The appliance fabrication system 150 can include appliance fabrication machinery 151, processor(s) 152, memory 153, and appliance generation modules 154. In general, the appliance fabrication system 150 can directly or indirectly fabricate aligners to implement a dental treatment plan. In some examples, the dental treatment plan may be stored in the treatment planning database(s) 135.

The appliance fabrication machinery 151 may include any feasible implement or apparatus that can fabricate any suitable dental aligner. The appliance generation modules 154 may include any non-transitory computer-readable instructions that, when executed by the processor(s) 152, can generate one or more design files that can correspond to stages determined by the staging modules 133. In turn, the one or more design files may be used to build or fabricate one or more dental aligners. In some examples, the appliance fabrication machinery 151 can use the design files to produce the one or more dental aligners. The memory 153 may store data or instructions for use by the processor(s) 152. In some examples, the memory 153 may temporarily store a dental treatment plan, dental models, or intraoral scans.

The computer-readable medium 160 (sometimes referred to as a non-transitory computer-readable storage medium) may include some or all of the elements described herein with respect to the dental computing environment 100. The computer-readable medium 160 may include non-transitory computer-readable instructions that, when executed by a processor, can provide the functionality of any device, machine, or module described herein.

FIG. 2 is a block diagram of one example of interactive user interface modules 200. The interactive user interface modules 200 may be wholly or partially included within the doctor system 120 of FIG. 1. The interactive user interface modules 200 may include a bite ramp user interface module 210, and an attachment collision interface module 220, and attachment-free user interface module 230. In general, the interactive user interface modules 200 can enable a user to modify one or more aspects of a patient's dental plan. For example, the interactive user interface modules 200 can enable a user to change an aspect of the dental treatment plan and visualize the effects of the changes on a display. In some cases, the interactive user interface modules can include a user interface (not shown for simplicity) to receive user inputs as well as display treatment planning results. The interactive user interface modules 200 can generate one or more views of a 3D digital model that correspond to updated dental treatment plan. In some examples, the updated treatment plan may be determined locally (within the interactive user interface modules 200) or at a remote server.

Notably, the operations performed by, or associated with the interactive user interface modules 200 provide a technical solution to a technical problem. Updating a dental treatment plan and generating a view of a 3D digital model showing predicted results of the updated treatment plan is a technical problem rooted in computer technology. Generating a view of the 3D digital model that predicts tooth movement based on a user modified dental treatment plan is a technical problem with a high level of difficulty. In most instances, it may be difficult and/or impossible to display a predicted view of the 3D digital model that is not time consuming or labor intensive. In addition, the technical solution provided by the interactive user interface modules 200 may provide a solution that is sufficiently efficient, particularly compared to a “brute force” method for maximizing modifying dental treatment plans and displaying predicted results of the modifications through 3D digital models.

The operations described herein provide a solution for a problem rooted in computer technology to overcome a problem specifically associated with interacting with a user to modify treatment plans, modifying the treatment plans, and simulating changes to tooth positions based on the modified treatment plans through a graphical user interface.

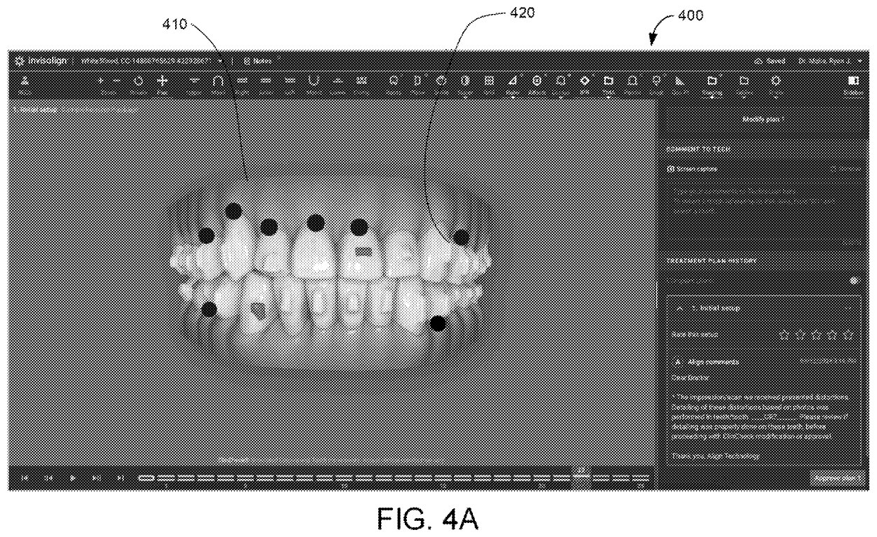

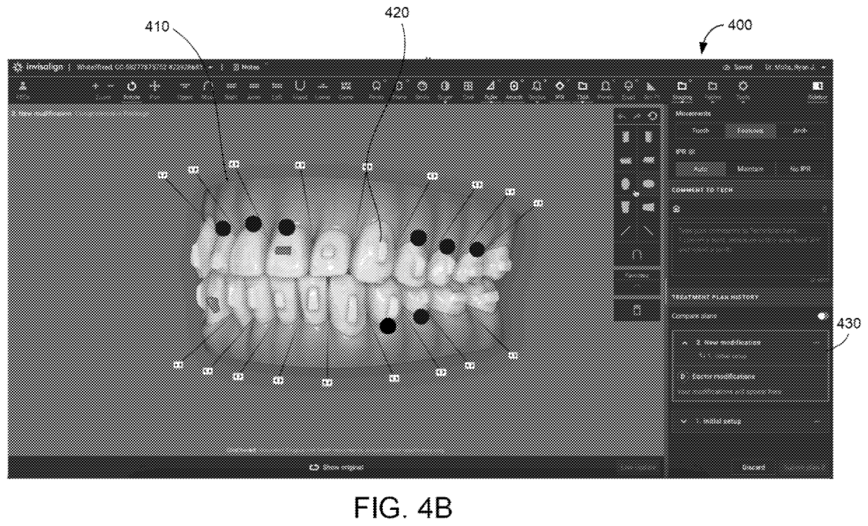

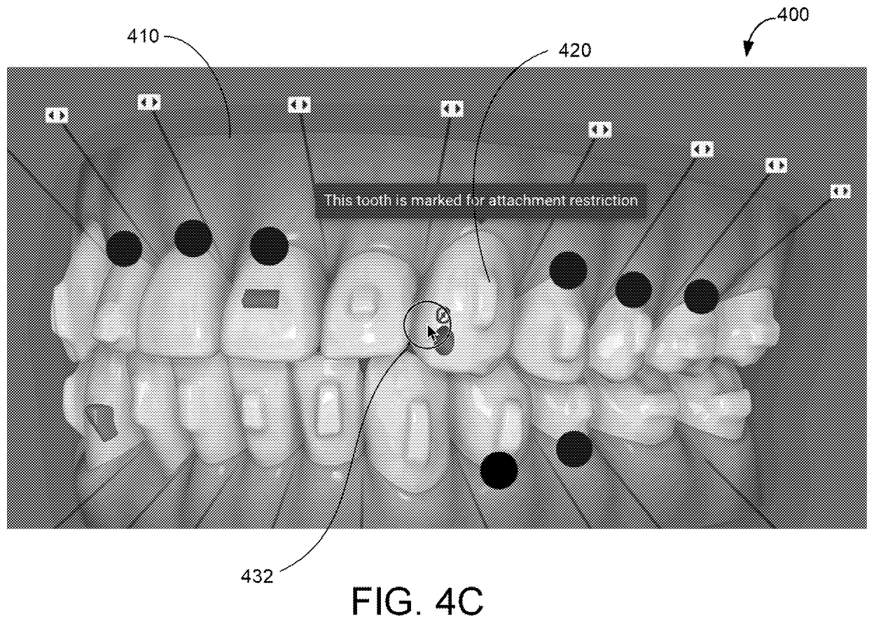

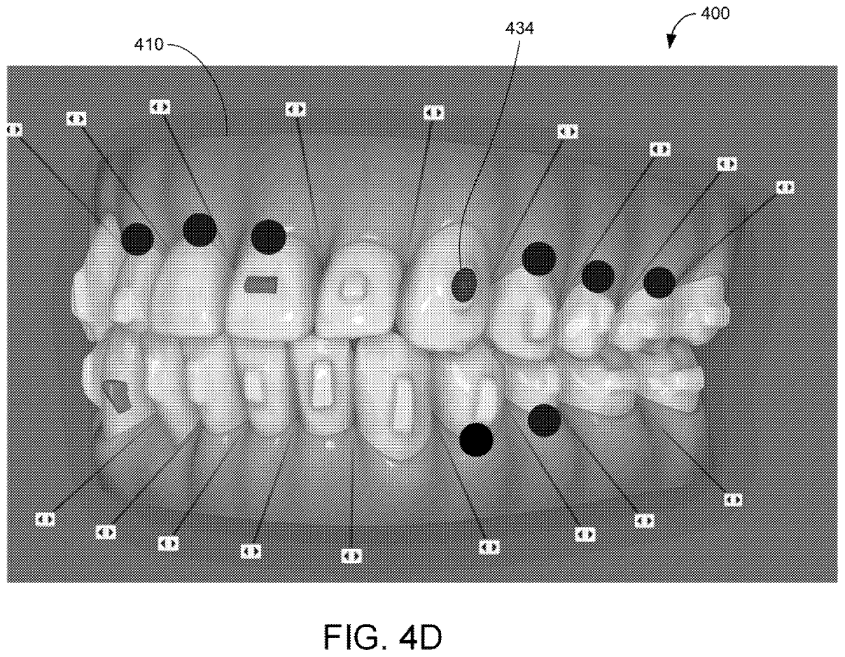

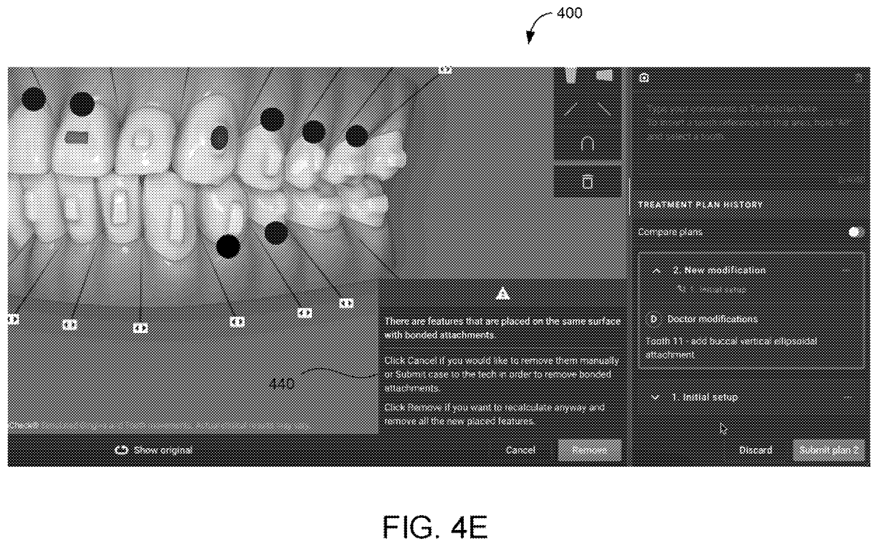

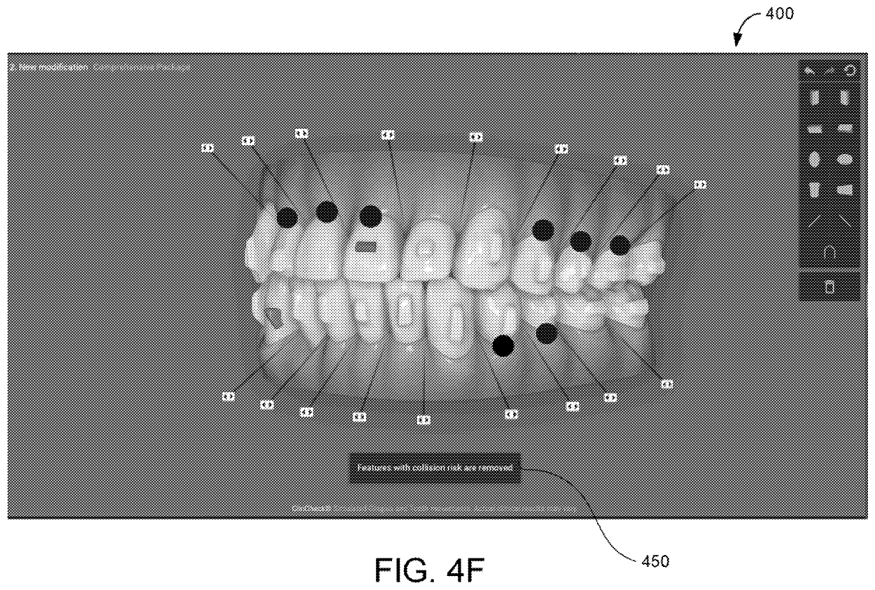

The bite ramp user interface module 210 can, through a user interface, receive inputs from a user to add or modify bite ramps to a dental aligner. In some examples, the bite ramp user interface module 210 can select one or more teeth to receive bite ramps (or any other feasible occlusal feature), update the 3D digital model, and recalculate the dental treatment plan and display a 3D digital model that includes updated (predicted) tooth positions based on the recalculated dental treatment plan. The bite ramp user interface module 210 is described in more detail with respect to FIGS. 4-6.

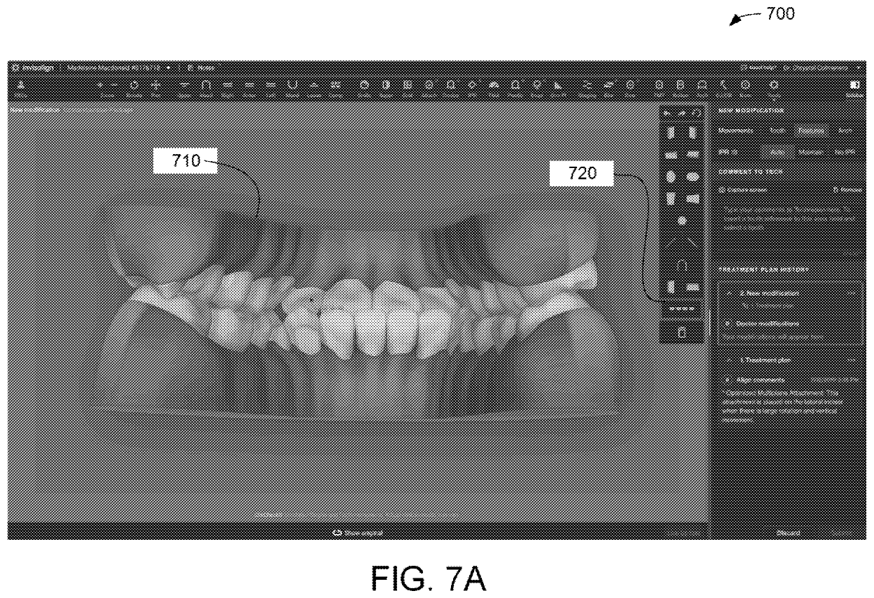

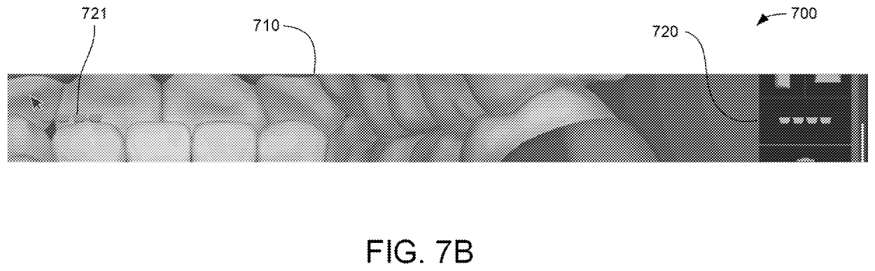

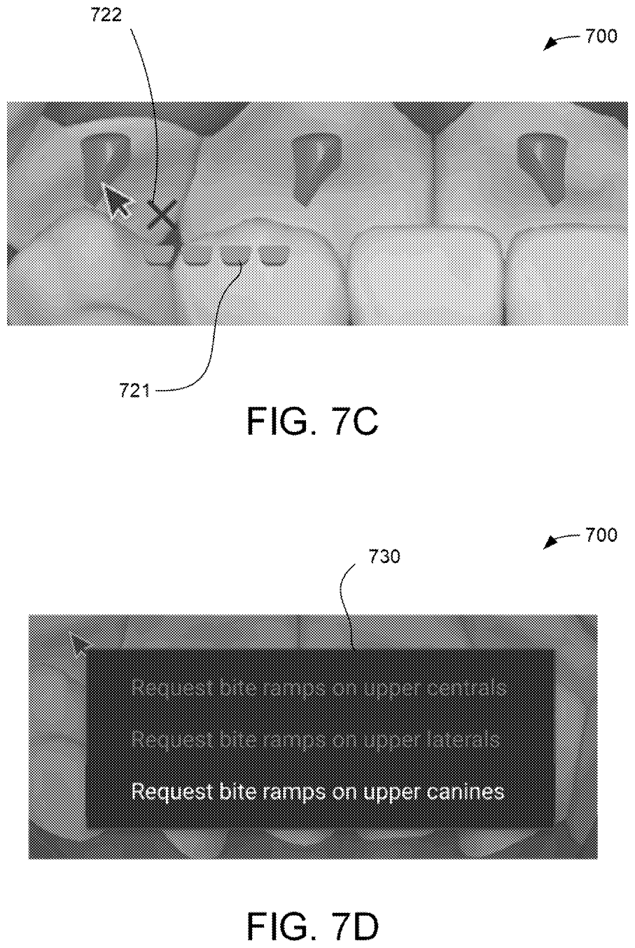

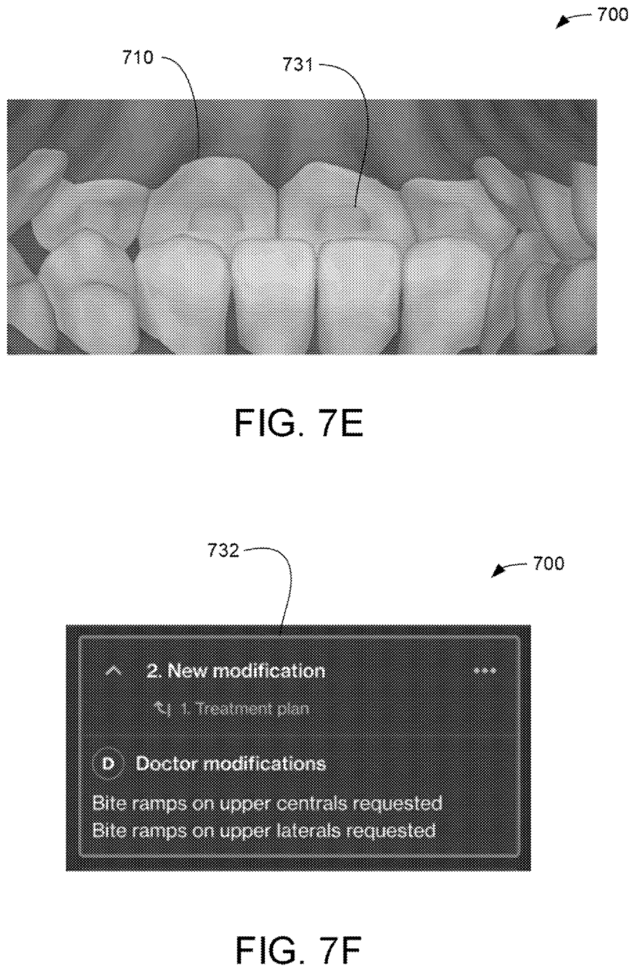

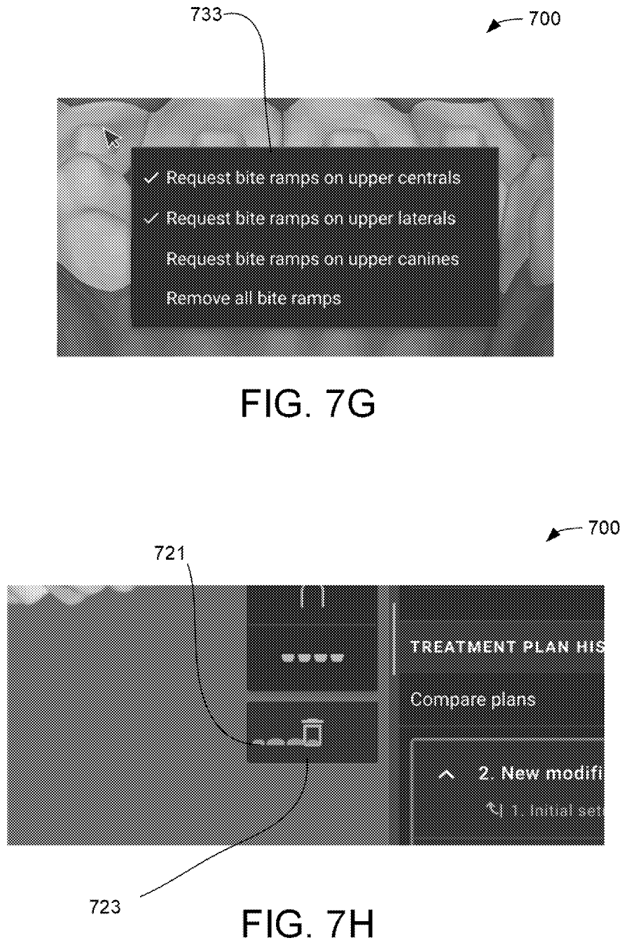

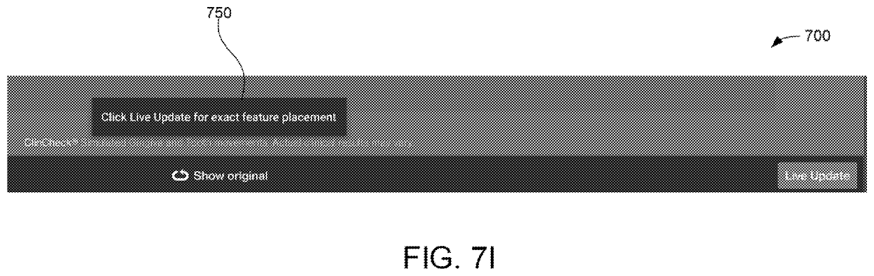

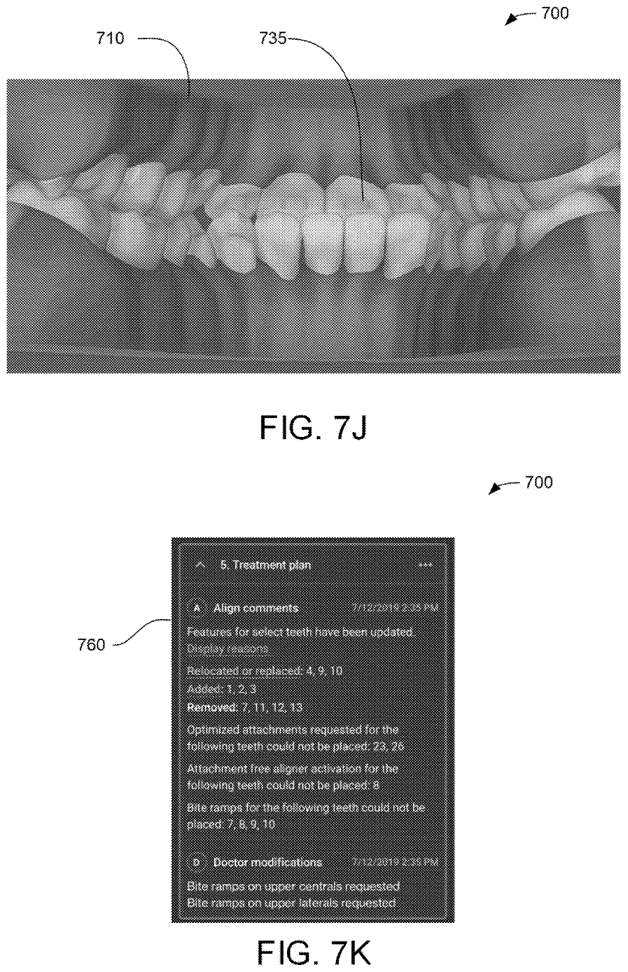

The attachment collision interface module 220 can, through a user interface, receive inputs from a user to add or modify an attachment to a tooth to implement a dental treatment plan. For example, after a dental treatment has begun, a clinician may rescan the patient's dentition to update the dental treatment plan. The attachment collision interface module 220 can detect whether the updated dental treatment plan specifies an attachment that conflicts with an existing dental attachment. The attachment collision interface module 220 can display conflicts on a user interface and interact with the user to address any collisions or interference. The attachment collision interface module 220 is described in more detail below with respect to FIGS. 7-9.

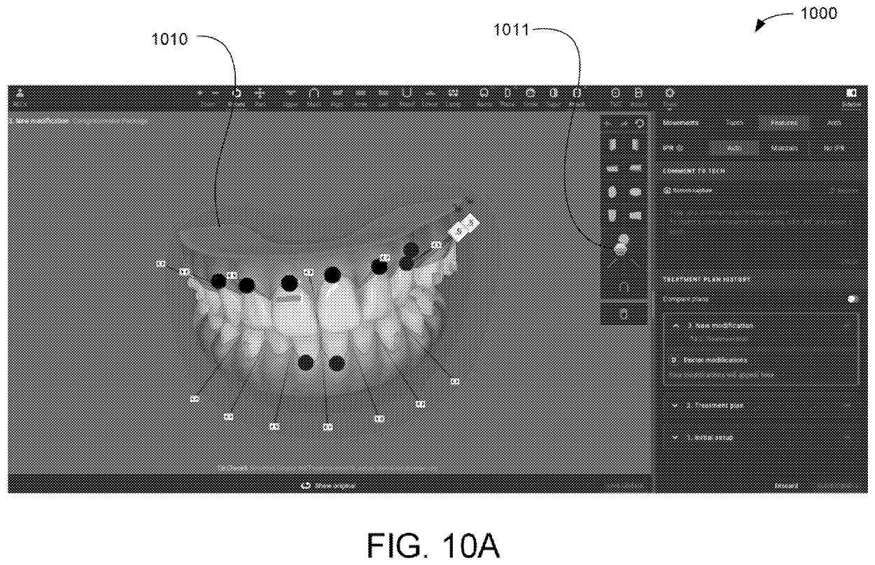

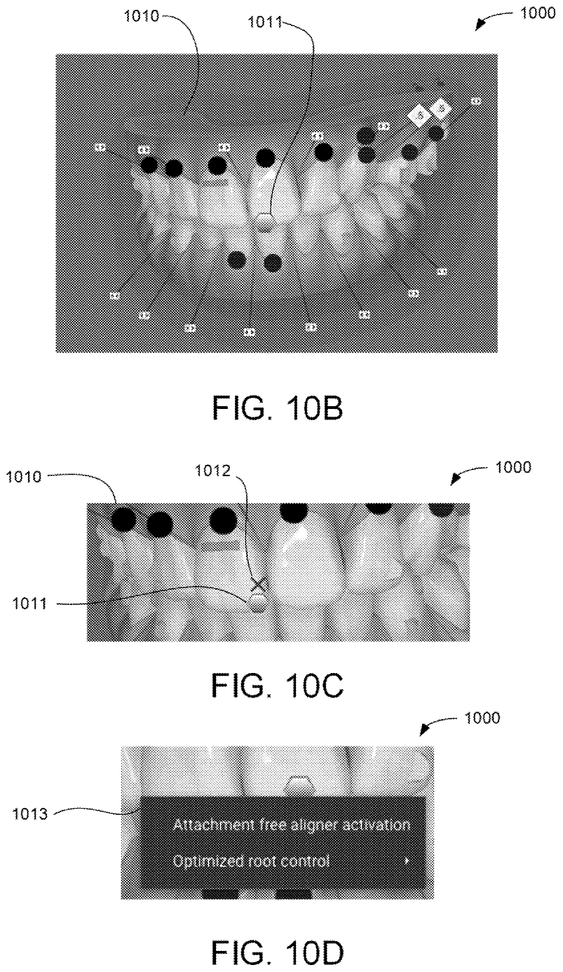

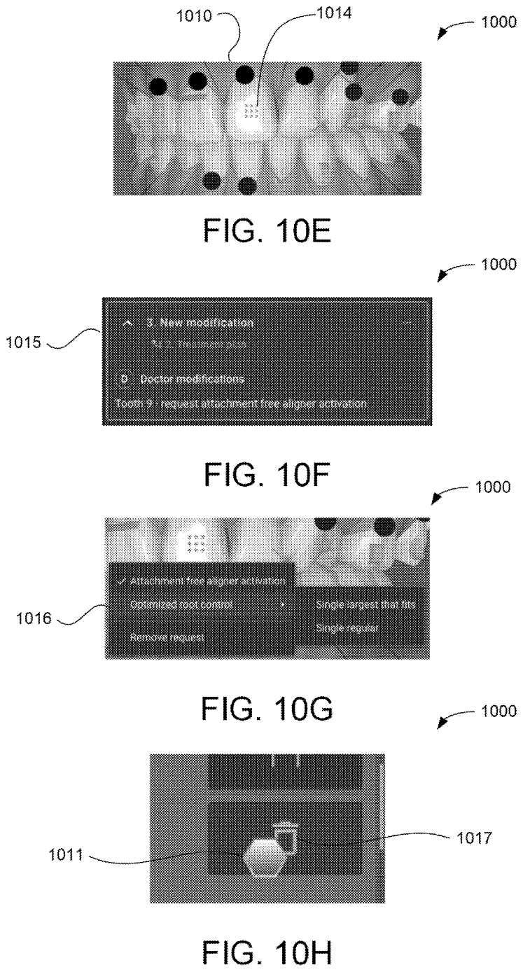

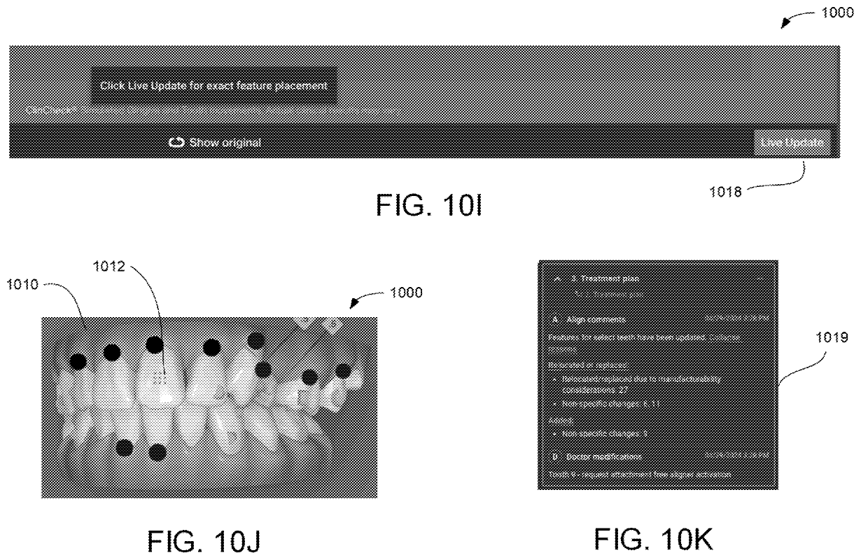

The attachment-free user interface module 230 can, through a user interface, receive inputs from a user to revise a dental treatment plan. In particular, the attachment-free user interface module 230 can revise a dental treatment plan by removing the use of dental attachments from some teeth. Additionally, the attachment-free user interface module 230 can simulate (and display) predicted tooth movements associated with the revised dental treatment plan. The attachment-free user interface module 230 is described in more detail below with respect to FIGS. 10-12.

FIG. 3 is a block diagram of one example of treatment planning modification modules 300. The treatment planning modification modules 300 may be wholly or partially included within the treatment planning system 130 of FIG. 1. In some examples, one or more elements of the treatment planning modification modules 300 may work and/or interact with one or more elements of the interactive user interface 123 of FIG. 1 or the interactive user interface modules 200 of FIG. 2. The treatment planning modification modules 300 may include a bite ramp treatment planning module 310, an attachment revision treatment planning module 320, and an attachment-free treatment planning module 330. In general, the treatment planning modification modules 300 can retrieve stages of a dental treatment plan (for example, stored in the treatment planning database(s) 135 of FIG. 1) and modify the dental treatment plan based on user inputs. In some cases, the user inputs may be provided by one or more modules within the interactive user interface modules 200.

The operations performed by or associated with the treatment planning modification modules 300 provide a technical solution to a technical problem. Updating a dental treatment plan and generating a view of a 3D digital model showing predicted results of the updated dental treatment plan is a technical problem rooted in computer technology. Generating a view of the 3D digital model that predicts tooth movement based on a user modified dental treatment plan is a technical problem with a high level of difficulty. In most instances, it may be difficult and/or impossible to display a predicted view of the 3D digital model that is not time consuming or labor intensive. The operations described herein provide a solution for a problem rooted in computer technology to overcome a problem specifically associated with interacting with a user to modify dental treatment plans, modifying the dental treatment plans, and simulating changes to tooth positions based on the modified dental treatment plans through a graphical user interface.

The bite ramp treatment planning module 310 can modify a dental treatment plan by accepting user inputs from the bite ramp user interface module 210 of FIG. 2. After receiving the user inputs, the bite ramp treatment planning module 310 can modify the dental treatment plan and predict tooth movements based on the user inputs. In some examples, a revised 3D digital model can be displayed to the user. The bite ramp treatment planning module 310 is described in more detail below in conjunction with FIGS. 4-6.

The attachment revision treatment planning module 320 can modify a dental treatment plan by accepting user inputs from the attachment collision user interface module 220. After receiving the user inputs, the attachment revision treatment planning module 320 can show, on a user interface, where new attachments may interfere with attachments that are already placed on the patient's teeth. The attachment revision treatment planning module 320 is described in more detail below in conjunction with FIGS. 7-9.

The attachment-free treatment planning module 330 can modify a dental treatment plan by accepting user inputs from the attachment-free user interface module 230 and revising a dental treatment plan by removing attachments from teeth that previously used attachments to control tooth movement, displacement, or affect aligner retention. The attachment-free treatment planning module 330 is described in more detail below in conjunction with FIGS. 10-12.

### Attachment Revision and Collision Detection

In some examples, a patient's dental treatment plan can be evaluated while a dental treatment plan is in progress. For example, a new dental scan of the patient's dentition can be performed and compared to a 3D model associated with the current dental treatment plan. The doctor (or other clinician) may decide to revise the dental treatment plan based on the new dental scan.

In some cases, the original (previous) dental treatment plan may have required that an attachment (sometimes referred to as aligner attachment) be placed on a tooth in order to attain the desired results in positions of the patient's teeth. In general, an attachment is a physical device that is temporarily affixed to a tooth that, in conjunction with a dental aligner, can be used to impart one or more orthodontic forces to the patient's dentition. However, during a re-evaluation of the dental treatment plan, the patient's dentition can be rescanned. The attachments used in the initial treatment now can show up on this dental scan (the rescan) as an existing attachment (at best) or something other than an attachment (at worst). In either case, when revising the dental treatment plan, the existing attachments can interfere with or collide with a new attachment that is associated with a revised dental treatment plan.

To assist the clinician (user) in revising the dental treatment plan, any interferences between existing and new attachments can be highlighted on a user interface. The user can address the interference by removing the old attachment and adding a new attachment, ignoring the old attachment, or in some cases utilizing the old attachment for the revised dental treatment plan.

FIGS. 4A-4F show a user interface 400 for use in the detection of interfering or colliding attachments. As mentioned above, in some examples, the progress of a patient's treatment may be evaluated while the treatment is ongoing. For example, a subsequent dental scan can be performed on the patient after treatment has already begun. In some cases, the patient may have one or more attachments affixed to one or more teeth based on an earlier dental treatment plan. FIG. 4A shows a 3D digital model 410 of the patient's dentition is shown. This 3D digital model 410 may be based on a rescan of the patient's dentition. The 3D digital model 410 can include previously used attachments 420, sometimes referred to as white attachments.

FIG. 4B shows the user interface 400 as the user interacts with the 3D digital model 410 to add a new feature to the treatment. In some examples, the user can interact with a “3D modification mode” menu 430 to make changes and, in some cases, access other menus in the user interface 400. Note that previously used attachments, such as attachment 420, are still shown on the 3D digital model 410.

FIG. 4C shows the user interface 400 as the user tries to place a new attachment at a location where a current attachment exists. The user interface shows the 3D digital model 410 and attachment 420. The user interface 400 can also show a user selected area 432 for a new attachment. The user interface 400 and/or associated system can determine that there is a potential interference between new and older attachments. In some examples, the user interface can provide a warning such as a pop-up indicating “This tooth is marked for attachment restriction,” or the like.

FIG. 4D shows the user interface 400 as the doctor ignores the warning regarding an interference. The user interface 400 shows the 3D digital model 410 and a visual indicator 434 at the area or region of a detected or determined interference between a new and an existing attachment. In some cases the visual indicator 434 can be rendered or displayed as a bright color to stand out in the user interface and show the user where the interference is located. For example, the visual indicator 434 may be rendered as a red, blue, orange, florescent green, or any other feasible color. After these or any other changes have been completed through the user interface 400, the user can select either “live update” or “submit.”

The live update selection will attempt to update the 3D digital model 410 to show the results of the user changes. In some examples, the user interface 400 will allow the user to select at least one of two options: 1) remove all new attachments that were placed on teeth that interfere with existing attachments or 2) cancel the “live update” and proceed with “submit.” FIG. 4E shows the user interface 400 with dialog boxes 440 that allow the user to remove placed attachments or proceed. By selecting “remove” all new attachments with collisions are removed. In some cases, a pop-up message 450 “Features with collision risk are removed,” is displayed and all original attachments are maintained. FIG. 4F shows a user interface 400 with a message indicating that features with collision risk are removed.

If the user wishes to proceed with new attachments (by clicking submit, not shown here), the desired changes to the 3D digital model 410 are sent to a remote server. A computer-aided design (CAD) designer can manually address any changes, in some cases by accepting the new attachments and deleting (from the 3D digital model 410) any old attachments that interfere with new attachments.

The methods and apparatuses described herein may use any appropriate technique for detecting interference between attachments. For example, these methods and apparatuses (e.g., systems) may determine interference between a first and second attachment by performing a direct geometric intersection analysis on their respective 3D meshes. Each attachment, whether an existing “white attachment” detected in a rescan or a proposed new “red” virtual attachment, may be represented within the 3D digital model as a polygonal mesh or volumetric surface. When the user places or drags a new attachment onto a tooth, the system may compute whether the polygons or surfaces of the new attachment intersect with the mesh of the existing attachment. If even a small region of overlap occurs-such as shared space, penetrating polygons, or mesh-to-mesh contact, e.g., the system may identify this as an interference. This form of geometric collision detection may be precise and may allow the system to determine whether two physical attachment shapes cannot coexist on the same tooth surface within the 3D model.

Alternatively or in addition to direct mesh intersection, the system may determine interference by evaluating the volumetric overlap between each attachment's bounding region. Each attachment may be associated with a bounding box, bounding sphere, or simplified collision hull that approximates its physical extent. When the new attachment is positioned, the system may check whether the bounding region intersects with that of the existing attachment by comparing spatial occupancy. If the regions overlap beyond a threshold, e.g., indicating two attachments would occupy the same physical space, the system may declare an interference. This volumetric method may be computationally efficient and allow the system to detect potential conflicts before running a more resource-intensive mesh-level collision test.

Any of these methods and apparatuses may also or alternatively determine interference without relying on geometric collisions by evaluating whether the user attempts to place two attachments on a surface of a tooth that is designated as exclusive. Existing attachments, particularly those identified in a rescan, may be associated with specific tooth surfaces such as the facial, lingual, mesial, or distal surfaces. If the user selects a location that falls within the same classified or reserved surface region as an existing attachment, the system may immediately recognizes a conflict. This rule-based interference detection may be grounded in the clinical reality that many surfaces cannot physically support two attachments simultaneously, even if their meshes do not strictly overlap in the digital model.

Any of these methods and apparatuses may use a minimum-spacing threshold between attachments. For example, the apparatus or methods may also or alternatively determine interference by enforcing a minimum required spacing between attachments. The treatment-planning rules may specify that attachments must be separated by at least a certain distance to avoid physical crowding, improper aligner engagement, or manufacturing complications. When a user attempts to place a second attachment, the system calculates the linear or surface distance between the proposed location and the centroid or boundary of the existing attachment. If the distance falls below a defined spacing threshold, the system may determine that the attachments cannot coexist and flags the placement as generating interference. This approach may ensure that even if meshes do not overlap, attachments still remain within clinically acceptable spacing constraints.

Any of these methods and apparatuses may use surface-normal and orientation-based constraint evaluation techniques. For example a method or apparatus as described herein may determine interference by analyzing the orientation of the tooth surface where the user attempts to place the new attachment. Each target region on a tooth surface has a surface-normal vector that indicates its outward orientation. Attachments may be restricted to surfaces with particular orientations to ensure proper aligner engagement. The system can detect interference by verifying whether the surface-normal region occupied by the proposed attachment is already allocated to an existing attachment or violates a rule governing orientation compatibility. If the new attachment would occupy an orientation zone already claimed by a white attachment, the system may identify that placement as an interference, even before considering geometric overlap.

Any of these methods and apparatuses may identify restricted zones based on white-attachment mapping. For example in scenarios where the patient has been rescanned during treatment, previously placed physical attachments appear on the scan as “white attachments.” These white attachments may become part of the tooth geometry and are mapped to specific surfaces or regions of the tooth. The system may use this mapping to identify restricted zones in which new attachments cannot be placed. When the user drags the attachment tool over a tooth, the system compares the proposed location to the stored white-attachment zones. If the user attempts to position a new attachment within such a restricted area, the system determines interference has occurred and presents a warning or restriction indicator. This method may help ensure clinical consistency between the digitally planned attachments and the physical attachments already bonded to the teeth.

These methods and apparatuses may use real-time constraint evaluation through CCBE first-approach logic. For example, another way the system may determine interference is through the ClinCheck 3D Scene Backend Component (CCBE), described herein, which pre-computes and provides real-time feasibility information for each tooth region. CCBE may apply a “first-approach logic” that identifies areas where attachments cannot be placed due to geometric, clinical, or prior-attachment constraints. As the user moves the cursor across the 3D model, CCBE may dynamically signal whether the location is permissible. If CCBE indicates that the region beneath the cursor is not allowed for attachment placement (e.g., because a white attachment exists there, because the geometry conflicts, or because a rule prohibits it) the system may identify this as interference. This early detection mechanism may prevent unnecessary processing and gives instant feedback to the user.

Any of these methods and apparatuses may use a backend treatment-planning engine (TPS) collision confirmation. For example, even after the local system detects potential interference, the backend treatment-planning engine (TPS) may perform a more authoritative collision analysis during a Live Update or Submit process. The backend may evaluate the proposed attachment placement in conjunction with clinical rules, planned tooth movements, and full aligner-stage geometry. When TPS calculates the updated treatment plan, it may determine whether the first attachment and the proposed second attachment are incompatible in the final design. If the backend determines that both attachments cannot be present, it may confirm interference and either removes the new attachment, removes the old white attachment, or provides conflict resolution instructions. This final stage collision detection may ensure fidelity between the clinician's changes and the final aligner design.

Any of these methods and apparatuses may use coordinate-space mapping and parameter comparison for attachment regions. The system may additionally (or alternatively) detect interference by comparing the coordinate-space definitions of attachment regions. Each attachment may be represented by a set of coordinates, anchor points, or parameterized shape descriptors stored relative to the tooth's coordinate system. When the user specifies a location for a new attachment, the system may map that location into the same coordinate space and evaluates whether the new coordinates fall within the boundary, tolerance range, or parameter footprint of an existing attachment. If the proposed coordinates overlap or conflict with the parameterized regions assigned to the first attachment, the system determines interference without needing a full mesh intersection test. This approach enables fast, precise detection based on structured position data.

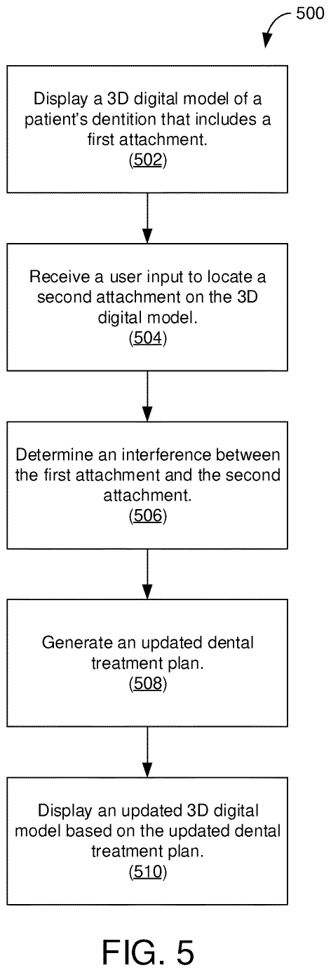

FIG. 5 is a flowchart showing an example method 500 for designing a dental treatment plan. Some examples may perform the operations described herein with additional operations, fewer operations, operations in a different order, operations in parallel, and some operations differently. The method 500 is described below with respect to the system 100 of FIG. 1, however, the method 500 may also be performed by any other suitable system or device.

The method begins in block 502 where a 3D digital model of a patient's dentition is displayed. In particular, the 3D digital model can include one or more attachments (a first attachment) disposed on one or more teeth. The 3D digital model may be displayed on any feasible user interface capable of showing the model and receiving user inputs. In some examples, the 3D digital model may be associated with a rescan (a subsequent scan) of the user's dentition. The rescan may be part of an assessment of progress of a patient's dental treatment plan.

Next, in block 504, the user can locate (position) a second attachment on the 3D digital model. For example, the user may wish to update or change an aligner design to update or change a desired tooth movement. The user may or may not be aware of the existing attachments in the 3D digital model.

Next, in block 506, the system 100 can determine that there is an interference between the new (second) attachment and the older (first) attachment. The interference can be caused by a collision or touching of the first and second attachment. In some cases, the interference may be caused by more than one attachment being placed on one (e.g., a common) tooth surface.

Next, in block 508, the system 100 can generate an updated dental treatment plan based on the determined interference. In some examples, the system 100 can show the user the potential collisions between attachments. The system 100 can perform a live update removing the attachments or the user can submit the design to a CAD designer where the interfering attachments are manually resolved. In either case, an updated dental treatment plan is generated that can include updated stages and associated aligners. The system 100 can also generate an updated associated 3D digital model.

Next, in block 510, an updated 3D digital model is displayed. The updated 3D digital model is associated with changes input by the user through the user interface. The user can review and/or approve changes to the dental treatment plan through the updated 3D digital model.

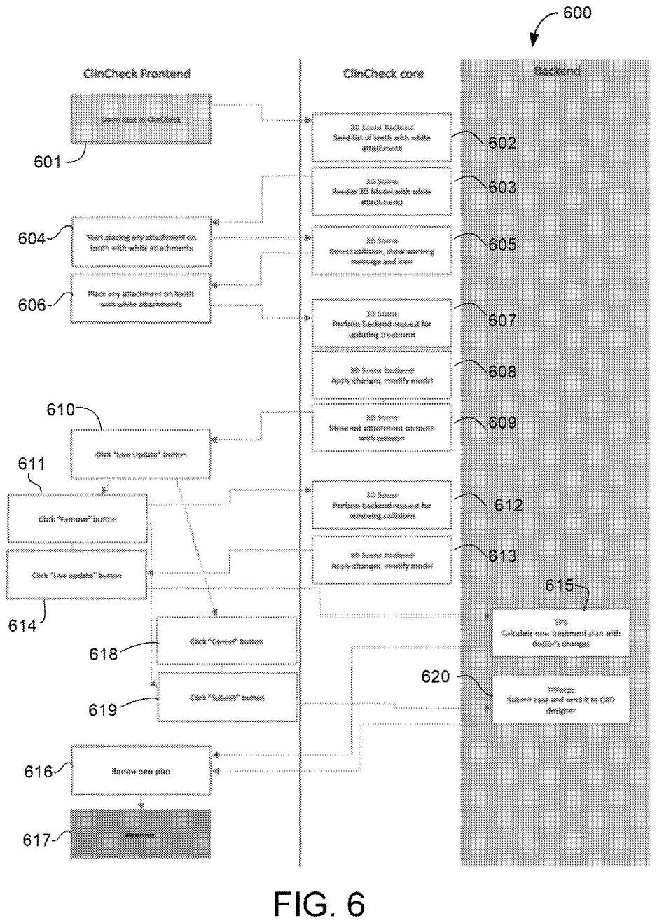

FIG. 6 is a flowchart showing an example method 600 for designing a dental treatment plan. The operations described with respect to FIG. 6 may be partially or wholly included within the operations described with respect to FIG. 5. The operations of FIG. 6 are described with respect to a ClinCheck frontend, a ClinCheck core, and a Backend. ClinCheck frontend and ClinCheck core refer to systems, methods, and/or apparatuses that are associated with the doctor system 120 of FIG. 1. The Backend refers to systems, methods, and/or apparatuses that are associated with the treatment planning system 130.

The method 600 begins in block 601 as a case is opened by a ClinCheck front end. Opening a case can refer to a user opening one or more files related to an existing dental treatment plan. The files may include treatment planning stages, related aligner design files, and related 3D digital models. Next, in block 602 the ClinCheck core can determine a list of teeth (referring in some cases by tooth numbers) that have attachments. Next, in block 603, the ClinCheck core can display a 3D digital model that includes the attachments. Next, in block 604, the ClinCheck front end can accept user inputs that place new attachments on teeth. For example, the user can place or position any number of attachments on a 3D digital model displayed on a user interface.

Next, in block 605 the ClinCheck core can detect attachment collisions (interferences) and can highlight the collisions/interferences and in some cases provide warning to the user regarding the collisions/interferences. For example, the ClinCheck core can detect and display warnings regarding collisions and interfaces on the user interface. Next, in block 606, the user can place an attachment such that it interferes with an existing attachment. Next, in block 607 ClinCheck core receive a request to update the dental treatment plan. In block 608, ClinCheck core can apply the user changes and modify the 3D digital model accordingly. Next, in block 609, ClinCheck core can show detected attachment collisions on the user interface.

Next, in block 610 the user can click on a “live update” button. The operations after the pressing the live update button can proceed with one of two possible outcomes. In a first outcome, the operation proceeds to block 611 where the user clicks the “remove” button. This action indicates that the user wishes to remove the new attachments from the revised dental treatment plan. Next in block 612 ClinCheck core receives the request to remove the attachments. The removal removes the interference, collision, and/or conflict between attachments. In block 613, ClinCheck core removes the attachments and updates the 3D digital model. Next, in block 614 the user can click the live update button. Next, in block 615, the Backend calculates a new/updated dental treatment plan with the user's changes. Next, in block 616 user can review the new/updated dental treatment plan. In some examples, the updated 3D digital modal is displayed on a user interface to allow the user to review the changes. Next, in block 617 the user can approve the changes.

The system may transfer the 3D digital model to the Backend where a treatment planning software can determine a new dental treatment plan based on the user's inputs in block 604. The method 600 continues to block 616 where the user is able to review the dental treatment plan. In some examples, a revised 3D digital model is displayed to the user on a user interface.

Returning to block 610, the method can proceed to block 618 as the user clicks the “cancel” button. The cancel button allows the user changes, including attachment collisions, to remain in the 3D digital model. Next, in block 619, the user clicks the “submit” button. This action submits the user inputs to the CAD designer and allows the CAD designer to review the collisions and resolve any discrepancies. Next, in block 620, the CAD designer receives the design files, the updated dental treatment plan and 3D digital model. The method proceeds to block 616.

Returning to block 610, the user clicks the “cancel” button in block 618. Then in block 619 the user can click the “submit” button. As a result, the user inputs are directed to the Backend in block 620. In block 620 the user inputs are analyzed and acted upon by a CAD designer to determine the revised dental treatment plan. The method continues to block 616.

### Occlusal Feature Insertion

Occlusal features can refer to any feasible features that may be a part of, or attached to, occlusal surfaces of teeth and/or dental aligners. In some examples, occlusal features may be used to correct a patient's bite. Conventionally, if an occlusal feature is a desired feature of a dental treatment plan, the user (clinician) submits a separate request to a CAD designer, often in the form of free-form text. The CAD designer would receive dentition data (3D digital model, and the like), the dental treatment plan data, and the request from the clinician. The CAD designer would then assess the dentition data and dental treatment plan data and manually insert the occlusal features into the design. This approach can be time consuming, particularly because there may be a non-deterministic amount of time before the CAD design can act on the inputs from the clinician. Further, the CAD designer may make unintentional errors when trying to implement the free-form text instructions. Finally, iterations of the design may be lengthy as well due to needing the subsequent results from the CAD designer.

In some examples, a clinician can add occlusal features to any dental treatment plan through a user interface, instead of through a conventional free-form text method. The user interface can show a 3D digital model of the patient's dentition. The clinician can select a location of the desired occlusal features through the user interface. The user interface can inform the clinician whether the occlusal features are possible/permitted on the selected locations. The patient's dental treatment plan can be updated according to the occlusal features and presented to the clinician for review and approval.

FIGS. 7A-7K show a user interface 700 for use in the addition and management of occlusal features into a dental treatment plan. In the examples of FIGS. 7A-7K, the occlusal feature is a bite ramp, however other occlusal features are contemplated. For example, an occlusal feature may include a disocclusion feature. In some examples, an occlusal feature may include a bite ramp shaped to disocclude a first jaw from a second jaw. In some other examples, an occlusal feature can include occlusal blocks that are placed on a lingual surface of anterior teeth. The occlusal blocks can disocclude one jaw from another. In still another examples, an occlusal feature can include a mandibular advancement feature. These features can be place on molars and can align one jaw to align against another in forward and backward directions. FIG. 7A shows a user interface 700 that includes a 3D digital model 710. To begin, the user interacts with the user interface 700, in some cases selecting a “3D modification mode.” After selecting the mode, a, occlusal tool may appear, shown in FIG. 7A as a bite ramps button 720.

FIG. 7B shows the user interface 700 which provides the user two ways to activate the bite ramps feature. In a first method, the user can toggle the bite ramps button 720. The user can then move the mouse to a desired location of the bite ramps and click the mouse through the user interface 700. In a second method, the user can click and drag the bite ramps button 720 to a desired location on the 3D digital model 710. A bite ramp tool icon 721 is shown at the desired location.

FIG. 7C shows the user interface 700 as the user drags or hovers over the bite ramp tool icon 721. If the bite ramps cannot be placed at a selected location, the user interface 700 can show an indicator 722. The indicator 722 is an X in this example.

FIG. 7D shows the user interface 700 showing possible placement options 730 that may be selected by the user. The user interface 700 can determine where and which bite ramps can be placed. Thus, the user interface 700 can advantageously reduce processing time without the need for resource-intensive services.

After the user selects one or more placement options (through placement options 730), the 3D digital model 710 is updated showing placeholder objects that can represent the placed bite ramps. For example, FIG. 7E shows the user interface 700 with placeholder bite ramps 731 on the 3D model 710. Additionally, data associated with the added bite ramp modifications may be shared with other programs, such as Backend programs. As shown in FIG. 7F, the user interface 700 may include user notifications 732 regarding placed occlusal features.

In some examples, the placeholder objects can be interactive with respect to the user. FIG. 7G shows the user interface 700 with a notification 733 showing choices that the user may have for the placed occlusal objects. In some cases, the placed bite ramps can be removed by clicking a trash icon. For example, FIG. 7H show the user interface 700 with a trash icon 723. A bite ramp tool icon 721 can be dragged to the trash icon 723 or clicked to remove the bite ramp feature.

At this stage, the placeholder bite ramps 731 may signify the user's intention to incorporate bite ramps into the dental treatment plan. The specific form, type, and positioning of the bite ramps may be finalized during a treatment recalculation process. In one example, an automated recalculation process within ClinCheck is referred to as Live Update. FIG. 7I shows the user interface 700 with a button 750 to begin recalculation of the dental treatment plan.

After recalculation of the dental treatment plan, the updated 3D model can be displayed to the user. For example, FIG. 7J shows the user interface 700 with an updated 3D digital model 710 showing recalculated bite ramps 735. In some examples, a messages or comments associated with the recalculation of dental treatment plan can be displayed for the user. FIG. 7K shows the user interface 700 that includes a window 760 with messages associated with the recalculation of the dental treatment plan.

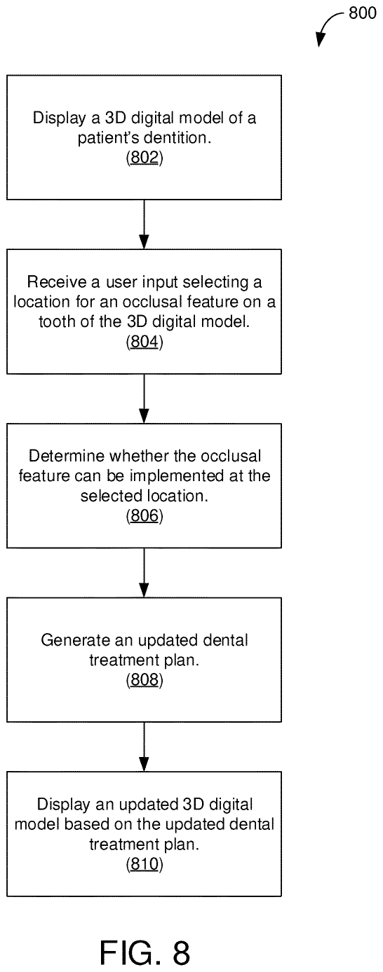

FIG. 8 is a flowchart showing an example method 800 for designing a dental treatment plan. The method 800 is described below with respect to the system 100 of FIG. 1, however, the method 800 may also be performed by any other suitable system or device.

The method 800 begins in block 802 as a 3D digital model of the patient's dentition is displayed. The 3D digital model can be associated with a patient's dental treatment plan and show occlusal surfaces associated with the patient's dentition. Next, in block 804 the system 100 receives a user input selecting a location for an occlusal feature on a tooth of the 3D digital model. In some examples, the user may use a user interface to select a location for the occlusal feature. In some cases, the user can select an occlusal surface of a tooth. The teeth may include incisors, canines, and the selected location can include lateral surfaces on the incisors and/or canines. In some examples, the user can click and drag an occlusal feature to a selected location. In some other examples, the occlusal feature can be selected simply by clicking on a location.

Next, in block 806, the system 100 determines whether the occlusal feature can be implemented at the selected location. In some examples, the occlusal features may be limited to canine and incisor teeth. Thus, if the user selects a tooth other than a canine or an incisor, the system 100 can indicate that the occlusal feature cannot be placed on the selected tooth. In some examples, an indication can be displayed on the user interface to inform the user that the occlusal feature cannot be placed at the selected location.

For example, these methods and apparatuses may determine whether an occlusal feature can be implemented using tooth-type eligibility and anatomical constraints. For example, the apparatus may determine whether an occlusal feature can be implemented by evaluating the anatomical characteristics of the tooth selected by the user. When the clinician clicks on a region of the 3D dental model to place a bite ramp or other occlusal feature, the system first identifies which tooth the user has selected, such as whether it is an upper incisor, upper canine, or another tooth type. The apparatus compares this tooth classification against a stored set of occlusal-feature eligibility rules. For instance, if a particular type of bite ramp is permitted only on upper incisors and canines, the system will determine that the feature cannot be implemented on a premolar or molar. If the selected tooth does not match a permitted category, the apparatus may immediately conclude that implementation is not feasible and provides visual feedback (e.g., an “X” icon or blocked placement indication). This determination avoids unnecessary calculations or server requests and ensures that occlusal features are placed only where clinically appropriate.

Alternatively or additionally, any of these methods and apparatuses may use surface-geometry and curvature analysis to determine whether an occlusal feature can be implemented. For example, the apparatus may evaluate whether the occlusal feature can be implemented by analyzing the geometric characteristics of the tooth surface at the selected location. When the user drags the occlusal-feature tool over the 3D digital model, the apparatus computes the surface curvature, surface normal vectors, and available bonding area at that specific location. Many occlusal features require a minimum flatness or curvature threshold to ensure proper aligner fabrication and functional occlusal contact. If the apparatus determines that the selected surface is too steep, too rounded, or oriented in a way that would prevent secure integration of the occlusal feature, it concludes that the feature cannot be applied. The system may then indicate this to the user through UI cues such as a highlighted red placement icon or tooltip warning. Only when the geometric analysis confirms a suitable surface does the apparatus allow placeholder objects to appear, indicating that implementation is feasible.

Alternatively or additionally, any of these methods and apparatuses may use occlusal-interaction and bite-relationship evaluation to determine whether an occlusal feature can be implemented. In some embodiments the apparatus determines implementation feasibility by evaluating how the proposed occlusal feature would interact with the opposing dentition during simulated occlusion. Certain occlusal features, such as bite ramps or disocclusion features, are intended to influence jaw relationships. Therefore, when the clinician selects a possible placement location, the apparatus consults clinical interaction rules to determine whether adding the feature at that specific location would appropriately modify the occlusal relationship without causing undesirable contact or interference. The apparatus may simulate a closing bite or approximate the opposing arch's position using stored occlusal models. If the simulation indicates that placing the occlusal feature would cause excessive interference, create an unstable occlusal contact, or contradict a prescribed bite-correction strategy, the apparatus determines that the feature cannot be implemented and informs the user accordingly.

Any of these methods and apparatuses may use treatment-Plan Compatibility and Movement Constraints to determine whether an occlusal feature can be implemented in addition to or instead of any of these techniques. For example, the apparatus may determine whether an occlusal feature can be implemented by analyzing the tooth's planned orthodontic movements across the treatment stages. Each tooth's movement trajectory, including intrusion, extrusion, tipping, torque, rotation, and translation, is part of the digital treatment plan. If the apparatus identifies that the tooth is scheduled for a movement that would be incompatible with the physical presence of a bite ramp, such as a movement requiring an unimpeded occlusal surface, then the system determines that the occlusal feature cannot be placed on that tooth. Similarly, if adding an occlusal feature would impede the aligner's ability to sit properly on the tooth during certain stages, the system will flag the placement as infeasible. This ensures that occlusal features are integrated only when they do not compromise planned biomechanical tooth movements.

Any of these methods and apparatuses may evaluate clearance for aligner fabrication to determine whether an occlusal feature can be implemented. The apparatus may also determine whether the occlusal feature can be implemented by simulating whether the aligner can be fabricated with sufficient material thickness and geometric fidelity around the feature. When the user proposes placement, the apparatus evaluates whether adding the occlusal feature would lead to thin aligner walls, unsupported material sections, or areas that exceed manufacturing tolerances. If the simulation indicates that the aligner cannot be fabricated reliably or that the occlusal feature would deform the aligner in a manner that compromises the patient's fit or comfort, the apparatus concludes that the feature is not implementable. A feasibility indicator is shown on the interface, preventing the user from finalizing an impractical placement.

In some example, rule-based validation using pre-computed occlusal constraints may be used to determine whether an occlusal feature can be implemented. For example, the apparatus may determine whether the occlusal feature can be implemented by comparing the selected placement location against a pre-computed occlusal constraint map. This map may identify tooth regions that are categorically disallowed for occlusal features due to clinical guidelines, prior aligner designs, previously placed attachments, or predicted inter-arch interactions. When the clinician interacts with the 3D model, the system queries this constraint map to instantly determine if the location is permitted. If the selected region is marked as restricted, the apparatus blocks placement and communicates to the clinician that the occlusal feature cannot be implemented there. This method enables fast, deterministic evaluation without the need for repeated server or CAD-designer interaction.

In some case, these methods and apparatuses may use live-update simulation using backend treatment recalculation to determine whether an occlusal feature can be implemented. In some cases, the apparatus may determine feasibility during a Live Update process performed by the backend treatment-planning system. After the clinician selects a placement location, the system transmits the request to the backend, which recalculates the treatment plan by incorporating the proposed occlusal feature. During this recalculation, the backend evaluates aligner shape, staging effects, biomechanical forces, occlusal contacts, and tooth movements to determine whether the feature is compatible with the overall treatment plan. If the recalculation fails, or produces a clinically unacceptable result, the backend returns data indicating the occlusal feature cannot be implemented. The UI then informs the clinician of the negative outcome. This approach provides the most authoritative determination since it evaluates feasibility in the context of the full treatment model.

Returning to FIG. 8, the system 100 can generate an updated dental treatment plan 808. The updated dental treatment plan can include the placement of the occlusal feature on the selected teeth.

Next, in block 810 the system 100 can display an updated 3D digital model. In some examples, the 3D digital model can include the occlusal features.

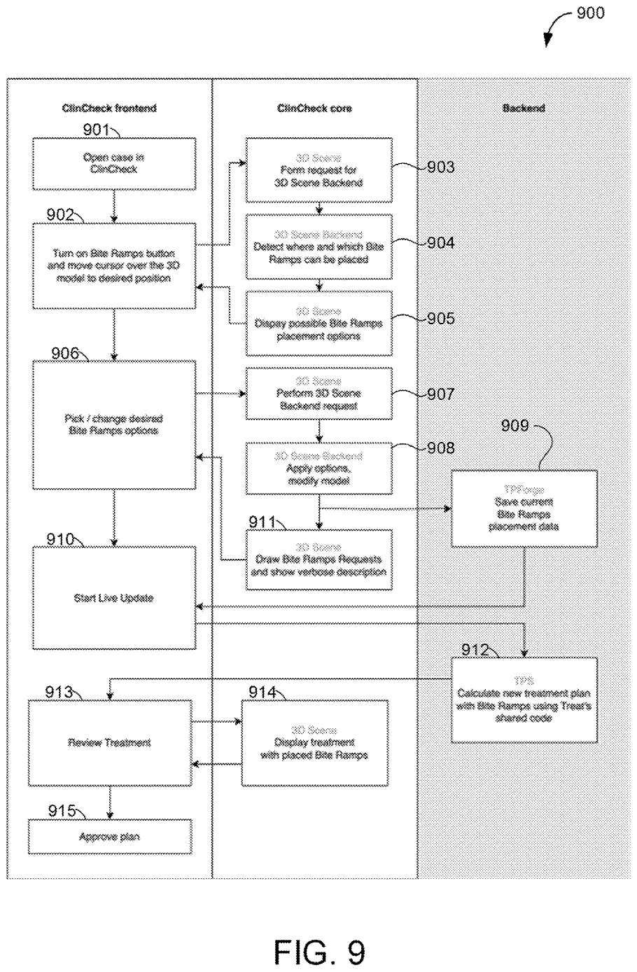

FIG. 9 is a flowchart showing an example method 900 for designing a dental treatment plan. The operations described with respect to FIG. 9 may be partially or wholly included within the operations described with respect to FIG. 8. The operations of FIG. 9 are described with respect to a ClinCheck frontend, a ClinCheck core, and a Backend. ClinCheck frontend and ClinCheck core refer to systems, methods, and/or apparatuses that are associated with the doctor system 120 of FIG. 1. The Backend refers to systems, methods, and/or apparatuses that are associated with the treatment planning system 130.

The method 900 begins in block 901 where a case is opened in ClinCheck. In some examples, opening a case can include retrieving an existing dental treatment plan and related dentition scan files for a patient. Opening a case can also include displaying a 3D digital model on a user interface.

Next, in block 902 the system 100 can display a bite ramps (occlusal feature) button on the user interface and permit the user to select a location for the bite ramps. In some examples, the user can use a mouse to move a cursor to select a surface of a tooth for the bite ramp.

Next, in block 903, the ClinCheck core can receive a request to display a 3D scene. The 3D scene can include a 3D digital model according to a dental treatment plan. Next, in block 904 the ClinCheck core detects locations on the 3D dental model that can receive any occlusal features, such as bite ramps. Next, in block 905 the ClinCheck core displays possible (allowable) locations for occlusal features. The operation flow returns to block 902.

Next in block 906 the user can select and/or change different occlusal feature options. The feature options can include a selected location of the occlusal features. The method 900 proceeds to block 907 where the ClinCheck core receives a request to display a 3D scene of the treatment plan. Next, in block 908 the ClinCheck core can apply the changes and selections to the 3D dental model. Next in block 909 the ClinCheck core can save the changes to the digital model and proceed to block 910.

Returning to block 911, the ClinCheck core can draw bite ramps and show a verbose description on a user interface. The method 900 can proceed to block 910. In block 910 a live update procedure can begin. Next in block 912, the backend can calculate a new dental treatment plan.

Next, in block 913 the user can review changes to the dental treatment plan. In some examples, the modified dental treatment plan may be shown on a user interface. Next, in block 914 the dental treatment plan is displayed included the new occlusal features. Next, in block 915 the user can approve the changes to the dental treatment plan.

### Aligner Activation Feature Insertion

Some dental treatment plans may benefit from aligner activation. Aligner activation can enable a user to update a dental treatment plan by eliminating the need for an attachment. As described herein, an attachment is an object affixed to a patient's tooth that enables the aligner to exert forces to the patient's tooth and control the tooth's displacement. The attachment generally engages with a corresponding well in the patient's aligner.

In some situations an attachment may be removed from a dental treatment plan as the aligner may be designed to exert effective forces on the tooth without the attachment. This process may be referred to as aligner activation. In some examples, a user can select a tooth through a user interface and check to see if an attachment-free solution is available. If there is an attachment-free solution available, the patient's dental treatment plan can be updated and displayed for the user to review.

FIGS. 10A-10K show a user interface 1000 for use in the identification of attachments which can be removed from teeth with respect to a patient's treatment plan. The examples of FIGS. 10A-10K show possible user interactions with the user interface 1000 to select and identify attachments that can be removed.

FIG. 10A shows the user interface 1000 displaying a patient's 3D digital model 1010. The user can navigate the user interface 1000 and select the “optimized attachment tool” 1011. In some examples, the 3D digital model 1010 may be associated with previous dental scans and/or an existing dental treatment plan. FIG. 10B shows the user interface 1000 including the 3D digital model 1010. The user drags the optimized attachment tool 1011 over regions of the 3D digital model 1010 to identify a location where aligner activation may be desired. In some examples, the user may use the optimized attachment tool 1011 to identify and/or select a location of an existing attachment or a location of a planned attachment.

FIG. 10C shows the user interface 1000 and the 3D digital model 1010. In this example, the user drags the optimized attachment tool 1011 to a location on a tooth. The user interface 1000 shows an indicator 1012 to indicate that aligner activation cannot be used at this location. In this manner, the user can receive relatively real-time feedback regarding the use of aligner activation at the selected location. If the user interface 1000 determines that the selected location is applicable for aligner activation, then a menu regarding possible placement options can be displayed. FIG. 10D shows an example menu 1013 regarding placement options on the user interface 1000. The menu 1013 is only displayed when the user has selected a valid location for selection by the optimized attachment tool 1011.

After selecting a valid location, the user interface 1000 can display a placeholder icon on the digital model. FIG. 10E shows the user interface 1000 and the 3D digital model 1010. A placeholder icon 1014 is displayed on the user selected location on the 3D digital model 1010. The user interface 1000 can also include text notifications regarding the aligner activation. For example, FIG. 10F shows a pop-up text box 1015 with descriptions and/or information associated with the aligner activation.

In some examples, the user may wish to modify the position or placement of the optimized attachment tool 1011. The user may click or tap on the placeholder icon 1014 and thereafter select a different location for aligner activation. In this manner the user interface 1000 can interact with the user. In some cases, if the user selects a location that cannot be used for aligner activation, then the user interface 1000 can again show the icon 1011. FIG. 10G shows the user interface 1000 another pop-up text box 1016 with additional information and selections for the user to interact with.

Aligner activation can also be removed. FIG. 10H shows a trash icon 1017 displayed on the user interface 1000. In some examples, the user can drag the optimized attachment tool 1011 to the trash icon 1017 to remove one or more aligner activation requests.

At this point, the placeholder icon 1014 can indicate the user's intention to use aligner activation to remove an attachment from the dental treatment plan. In some cases, the specific position of the aligner activation solution is only finalized during a treatment recalculation process. Recalculation may be referred to as a Live Update. FIG. 10I shows the user interface 1000 along with an interactive button 1018 to allow the user to perform a Live Update.

The Live Update operation can cause the dental treatment plan to be recalculated. FIG. 10J shows the user interface 1000 displaying an updated 3D digital model 1010. Using the 3D dental model 1010, the user can review the predicted changes to the patient's dentition. Note, the position of the aligner activation can be indicated with the icon 1011. FIG. 10K shows a pop-up text box 1019 displaying feedback regarding the aligner activation to the user.

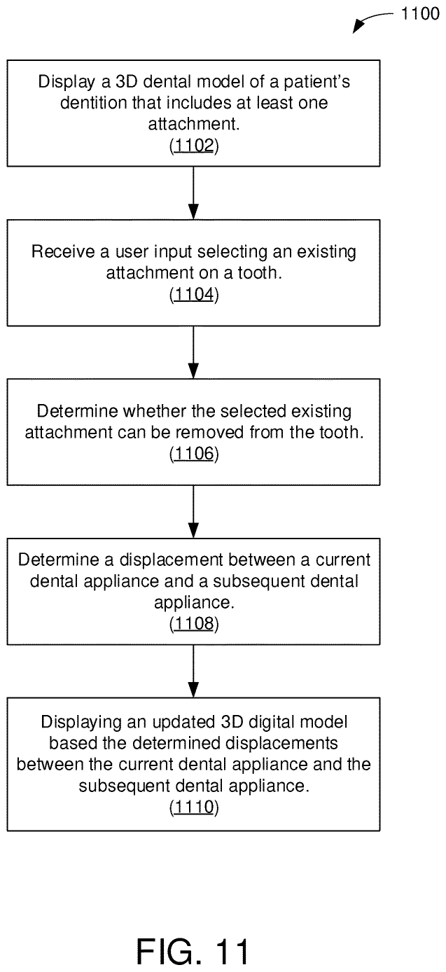

FIG. 11 is a flowchart showing an example method 1100 for designing a dental treatment plan. The method 1100 is described below with respect to the system 100 of FIG. 1, however, the method 1100 may also be performed by any other suitable system or device.

The method begins in block 1102 as a 3D digital model of a patient's dentition (sometimes referred to as a 3D dental model) that includes at least one aligner attachment (sometimes more simply referred to as an attachment). In some examples, the 3D digital model may be displayed on a user interface. In many cases, the 3D digital model is associated with a dental treatment plan. The dental treatment plan, in turn, can be associated with a plurality of stages where each stage can be associated with a dental aligner. In some cases, the 3D digital model can include locations of at least one (or any feasible number of) aligner attachments. Any attachment shown on the 3D digital model may have a corresponding well in any dental aligner associated with the same dental treatment plan. The attachment can engage with the corresponding well enabling the aligner to provide directed orthodontic forces to particular teeth.

Next, in block 1104 the system 100 receives a user input selecting an existing aligner attachment on a tooth. For example, the user can interact with the 3D digital model and, through the user interface, select an existing aligner attachment. The existing aligner attachment can be associated with an existing dental treatment plan. The existing aligner attachment can also be associated with a location on a tooth and/or a particular dental aligner.

Next, in block 1106, the system 100 determines whether the selected existing attachment can be removed from the tooth. In some examples, the system 100 examines the planned displacement (tooth movements) of the tooth. Then, based on the amount of planned displacement, the system 100 can determine if the tooth movement can be accomplished with just an aligner (aligner activation) instead of using an attachment and a corresponding well in the aligner that engages with the attachment.

In some implementations, an amount of planned tooth intrusion can be used to determine if the planned tooth movement can be performed without an attachment. For example, there is less than 0.5 millimeters (mm) of planned tooth intrusion (this includes any value less than 0.5 mm, including 0.0 mm), then at least a single attachment is required to perform the planned tooth movement. On the other hand, if there is more than 0.5 mm of planned tooth intrusion, then no attachment is necessary to perform the planned tooth movement. In some other examples, the amount of total displacement for the tooth may be used to determine if the planned tooth movement can be performed without an attachment. In still other examples, the type of planned tooth displacement may be used to determine if the planned tooth movement can be performed without an attachment. The system 100 can determine not only Cartesian displacement, but also polar displacement, radial displacement or any other feasible displacement.

In other examples, the planned tooth movement may be based on a tooth root's center mesial or distal movement. For example, if the planned movement of the tooth root's center mesial of distal movement is greater than a predetermined amount, then tooth movement may be accomplished with aligner activation (no attachment is needed). In these cases, the crown center may be stationary in a mesial-distal direction, while the root center moves mesially or distally. In some other cases, the crown center may move not more than 0.25 mm along a mesial-distal direction.

In some cases, the root's center movement may be constrained within a cone that includes a region around a Y-axis of the tooth and a 60 degree angle (e.g. a 60 degree angle with respect to a Y-axis of the tooth where the Y-axis passes through the center of the tooth). Thus, if the root's center is constrained in this manner, then aligner activation may be used.

In some examples, the system 100 can also indicate to the user if the selected aligner attachment cannot be removed. In this way, the user can be informed that the alignment attachment is required and cannot be removed. In some other examples, the system 100 can indicate that the specified aligner attachment can continue to be used.

In some other implementations, only attachments on particular teeth may be candidates for removal (aligner activation). For example, aligner activation may be limited to upper central and/or upper lateral teeth. In other examples, the aligner activation can be limited to any one or more particular teeth.

In some examples, the system 100 can update the dental treatment plan and/or the 3D digital model based on a removal of the aligner attachment. In some other examples, the system 100 can send or transmit the user inputs to remove a selected aligner attachment to a remote computing system. The remote computing system can then determine if the aligner attachment can be removed and, in some cases, determine an updated 3D digital model. The 3D digital model can be sent to the user for review. In some examples, the system 100 can send or transmit the user inputs and the 3D model to a CAD designer to see if the aligner attachment can be removed.

Next, in block 1108, the system 100 determines a displacement between a current dental appliance and a subsequent dental appliance. The displacements between dental appliances are based on the planned tooth movements of the patient's dental treatment plan. In some examples, the sum of the tooth displacements of all the dental appliances can be approximately equal to the tooth displacements of the patient's dental treatment plan.

Next, in block 1110, an updated 3D digital model is displayed. The updated 3D digital model can be based on the determined displacements between the current and subsequent dental appliances. The user can use the displayed 3D digital model to review the overall dental treatment plan. In some cases, the updated 3D digital model is displayed on a user interface.

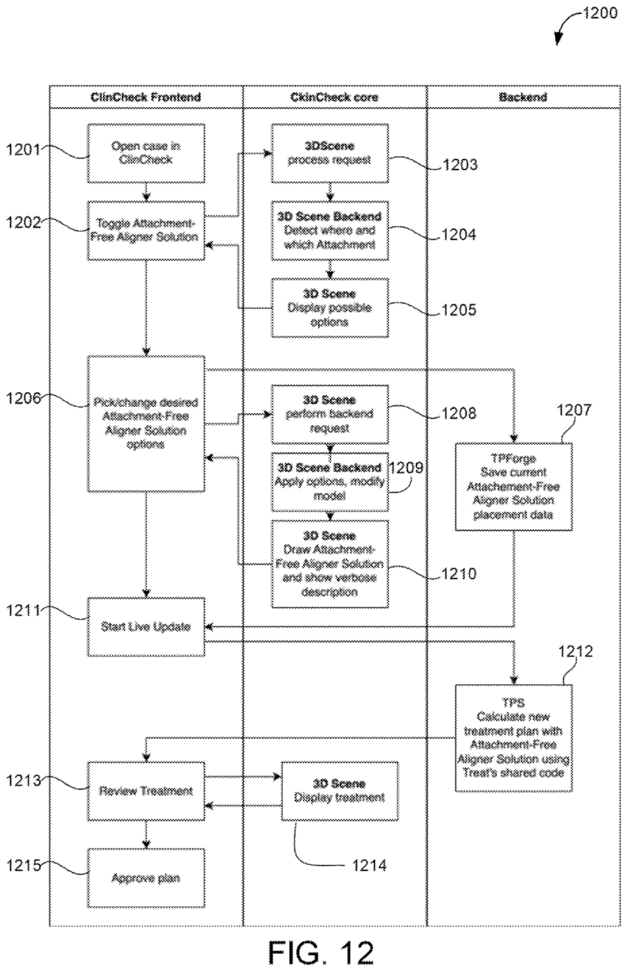

FIG. 12 is a flowchart showing an example method 1200 for designing a dental treatment plan. The operations described with respect to FIG. 12 may be partially or wholly included within the operations described with respect to FIG. 11. The operations of FIG. 12 are described with respect to the system 100 of FIG. 1. In some cases, the operations mention a ClinCheck frontend, a ClinCheck core, and a Backend. ClinCheck frontend and ClinCheck core refer to systems, methods, and/or apparatuses that are associated with the doctor system 120 of FIG. 1. The Backend refers to systems, methods, and/or apparatuses that are associated with the treatment planning system 130.

The method 1200 begins in block 1201 where a case is opened in ClinCheck. In some examples, opening a case can include retrieving an existing dental treatment plan and related dentition scan files for a patient. Opening a case can also include displaying a 3D digital model on a user interface.

Next, in block 1202 the user can toggle an attachment-free aligner solution. This action can include the user interacting with a user interface to enable determine whether an attachment-free aligner solution is feasible or possible with the case opened in block 1201.

Next, in block 1203 a request to process the 3D digital model is received. Next, in block 1204 the ClinCheck core determines the location of all aligner attachments with respect to the 3D dental model. Next in block 1205, the 3D digital model is displayed on the user interface. In some cases, aligner attachments can be highlighted on the display.

Next, in block 1206 the user can pick or select an aligner attachment. The user can select an attachment to determine whether a related attachment free solution is possible for the 3D digital model. The method 1200 then proceeds with one of two operations. In a first operation, in block 1207 the Backend saves a current attachment-free aligner solution. The method then proceeds to block 1211.

Returning to block 1206, in an alternative operation, in block 1208 a request is made to remove an aligner attachment. Next, in block 1209 the 3D digital model is modified for a possible removal of an aligner attachment. Next, in block 1210 the 3D digital model is displayed without the aligner attachment. The method then proceeds to block 1211.

In block 1211, the user selects a live update to begin. During the live update, in block 1212, a new (updated) dental treatment plan is determined based on the user request to determine an attachment-free solution. In some cases, the updated dental treatment plan is based on the amount of tooth displacement needed to accomplish a desired final position of teeth. In some examples, the 3D digital model can be updated according to the updated dental treatment plan.

Next, in block 1213, the user can review the dental treatment plan. In block 1214, the updated 3D digital model is displayed. In block 1215, the user approves the dental treatment plan based on the displayed 3D dental model.

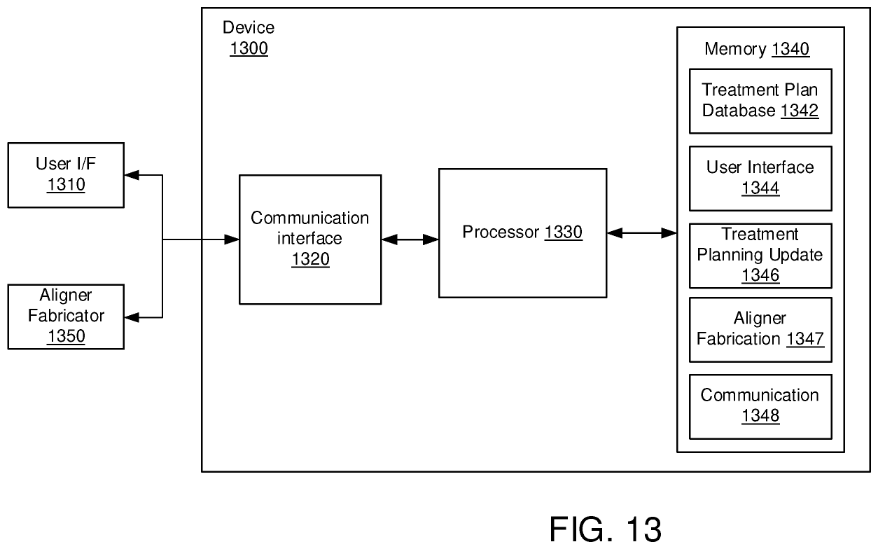

FIG. 13 shows a block diagram of a device 1300 that may be one example a device configured to perform one or more operations associated with the system 100 of FIG. 1. The device 1300 may include a communication interface 1320, a processor 1330, and a memory 1340.

The communication interface 1320, which may be coupled to a communication network (not shown) and to the processor 930, may transmit signals to and receive signals from other wired or wireless devices, including remote (e.g., cloud-based) storage devices, cameras, processors, compute nodes, processing nodes, computers, mobile devices (e.g., cellular phones, tablet computers and the like) and/or displays. For example, the communication interface 1320 may include wired (e.g., serial, ethernet, or the like) and/or wireless (Bluetooth, Wi-Fi, cellular, or the like) transceivers that may communicate with any other feasible device through any feasible network. In some examples, the communication interface 1320 may receive dental data (3D digital models), dental treatment plans, and treatment databases that are associated with a patient's dental treatment plan.

The communication interface is also coupled to a user interface 1310 and an aligner fabricator 1350. The aligner fabricator 1350 fabricates dental aligners based on a patient's dental treatment plan. In some examples, the aligner fabricator 1350 can fabricate one dental aligner for each stage of the dental treatment plan as provided by the staging modules 133 of FIG. 1.

The user interface 1310 is used to display information to the user and also receive user inputs to guide execution of one or more programs or modules. For example, the user interface 1310 can include a display to show a 3D digital model of the patient's dentition as well as buttons and menus for the user to interact with to guide the execution of various software module and/or programs. The user interface 1310 can include a keyboard, mouse, touchscreen and the like to receive user inputs.

The processor 1330, which is also coupled to the memory 1340, may be any one or more suitable processors capable of executing scripts or instructions of one or more software programs stored in the device 1300 (such as within memory 1340).

The memory 1340 may include treatment plan database 1342 that may be used to locally store patient treatment plans and related 3D digital models. For example, the treatment plan database 1342 may include one or more 3D digital models for patients that may be based, at least in part, on dental treatment plans for that patient. In some cases, the 3D digital models may be received through the communication interface 1320 from remote servers, compute nodes, or the like.

The memory 1340 may also include a non-transitory computer-readable storage medium (e.g., one or more nonvolatile memory elements, such as EPROM, EEPROM, Flash memory, a hard drive, etc.) that may store the following software modules: a user interface module 1344 to determine transmit and receive data with the user interface 1310; an treatment planning update module 1346 to determine, modify and/or generate a dental treatment plan; an aligner fabrication module 1347 to generate dental appliance data in accordance with a dental treatment plan; and a communication module 1348 to transmit and receive data through the communication interface 1320.

Each software module includes program instructions that, when executed by the processor 1330, may cause the device 1300 to perform the corresponding function(s). Thus, the non-transitory computer-readable storage medium of memory 1340 may include instructions for performing all or a portion of the operations described herein.

The processor 1330 may execute the user interface module 944 to display to the user visual information associated with one or more programs or operations performed by the device 1300. Additionally, the user interface module 944 can receive user input, oftentimes in response to data or images shown on a display of the user interface module. In some examples, the user interface module 1344 can display 3D digital models, menus, information boxes associated with detecting interfering or colliding attachments, adding occlusal features, and/or managing aligner activation as described herein. The user interface module 1344 can receive user input to manage or remove interfering or colliding attachments, adding occlusal features, and/or removing attachments and managing aligner activation. Thus, the user interface module may be used to perform some or all of the operations and methods described with respect to FIGS. 1-12.

The processor 1330 may execute the treatment planning update module 1346 to update a patient's treatment plan. For example, execution of the treatment planning update module 1346 may cause the processor 1330 to update the patient's treatment plan based on detecting interfering or colliding attachments, adding occlusal features, and/or managing aligner activation as described herein. Execution of treatment planning update module 1346 may also cause the display and/or user interface to show updated views of the patient's 3D digital model. In some examples, execution of the treatment planning update module 1346 may store the updated dental treatment plan and updated 3D digital model in the treatment plan database 1342.

The processor 1330 may execute the aligner fabrication module 1347 to generate a patient's dental appliance data. In some examples, the patient's dental appliance data may be determined by the appliance fabrication system 150 of FIG. 1. The dental appliance data may be transmitted to the aligner fabricator 1350 through the communication interface 1320.

The processor 1330 may execute the communication module 1348 to communicate with any other feasible devices. For example, execution of the communication module 1348 may enable the device 1300 to communicate via cellular networks conforming to any of the LTE standards promulgated by the 3^{rd} Generation Partnership Project (3GPP) working group, Wi-Fi networks conforming to any of the IEEE 802.11 standards, Bluetooth protocols set forth by the Bluetooth Special Interest Group (SIG), Ethernet protocols, or the like. In some embodiments, execution of the communication module 1348 may enable the device 1300 to communicate with cloud-based servers, network coupled displays, or other computer devices and/or the user interface 1310. In some other embodiments, execution of the communication module 1348 may implement encryption and/or decryption procedures.

In some cases, a SmartForce feature, attachment-free aligner activation, is the newest innovation as part of the Align digital workflow, that helps minimize the number of attachments while maintaining predictable treatment outcomes. Previously, doctors had to use optimized attachments that are small tooth-colored shapes attached to teeth and are designed to deliver precise forces when and where needed to achieve more predictable tooth movements. Double attachments are often needed on upper central (front) teeth to achieve desired mesiodistal root movements. Although optimized attachments are crucial for successful Invisalign treatment.

Now, with this new feature, certain movements now can be achieved using a similar force system through aligner activation, reducing the need for double attachments on the centrals. If an attachment is still needed for certain scenarios, aligner activation will be present with a single optimized root control attachment as with other teeth such as canines and premolars. Also, this innovation introduces a new workflow for Attachment-Free Aligner Activation placement, encompassing new UI/UX elements for requesting and displaying feedback, alongside changes in data flow to accommodate TPS recalculation capabilities.

The new SmartForce feature, Attachment-Free Aligner Solution, helps minimize the number of attachments while maintaining predictable treatment outcomes. Upgraded TPS now provides a recalculated treatment plan with an Attachment-Free Aligner Solution by utilizing the existing codebase shared with Treat for automated treatment plan recalculation. Extended data flow between TPS and ClinCheck allows the transfer Attachment-Free Aligner Solution request and result data. ClinCheck CCBE component adopts logic that allows to provide immediate feedback about Attachment-Free Aligner Solution placement possibilities. New UI/UX components in ClinCheck enable interactive placement of Attachment-Free Aligner Solution and visually display where and how they can be placed. Improved UI/UX components in ClinCheck highlight the outcome of Attachment-Free Aligner Solution requests effectively.

Some examples described herein can provide a no-attachments solution that allows certain movements that could be achieved only using a system with small attachments on the teeth. Reduced time delivery treatment with applied Attachments-Free Aligner Solution to patient. Helps to avoid misunderstanding and human factor between doctor and technician. Reduces the number of iterations needed to produce approvable treatment plan.

Automated placement of Attachment-Free Aligner Solution is now accessible within the ClinCheck application. However, this feature may be disabled for certain doctors and/or doctor groups based on factors such as product type, case type, or other clinical or regulatory criteria. A general flow diagram is provided at the conclusion of this section. Software shall place Attachment-Free Aligner Solution on upper incisors when the following triggers are satisfied. Note that the measurement of movement direction and amount is based on the tooth's basis at its final stage.

A new Single Attachment MDRC solution utilizing one attachment, pressure points, bubbles and Aligner Track Activation shall be introduced for Centrals and Laterals. A new Attachment Free MDRC solution utilizing Aligner Track Activation for upper Centrals and pressure points, bubbles, Aligner Track Activation for upper Laterals shall be introduced. Single Attachment MDRC solution shall be placed by default on an upper central or lateral tooth if there is MDRC movement and no intrusion (?) more than 0.5 mm planned for the tooth (<0.5 of intrusion). Attachment Free MDRC solution shall be placed by default on an upper central or lateral tooth if there is MDRC movement and intrusion more or equal to 0.5 mm planned for the tooth (>=0.5 of intrusion). New Single and Attachment Free MDRC solution shall be placed by default when applicable on upper Centrals and Laterals instead of the existing Dual Attachment MDRC solution for Upper Centrals and Single Attachment MDRC solution for Upper Laterals. Only compatible features shall be placed on a tooth with Attachment Free MDRC solution.

I To initiate the Attachment-Free Aligner Solution process, the doctor must navigate to the “3D Modification Mode” within the ClinCheck application and access the Features panel and click “Optimized attachment” tool. Then, move the cursor to the desired location on the 3D model and click again. While dragging or hovering over the “Optimized attachment” tool, ClinCheck provides the doctor with feedback regarding whether the Optimized attachment can be placed at the current position. If Optimized attachment cannot be placed at the current position, a red cross will appear along with the cursor. Once the doctor drops the “Optimized Attachment” tool to the desired location or clicks on the desired location when the “Optimized Attachment” tool is active, a menu with possible placement options will appear.

The ClinCheck 3D Scene backend component (CCBE) supplies data regarding the possibility of placing Attachment-Free Aligner Solution on the teeth below the current cursor position. It employs a first-approach logic, determining where and which Attachment-Free Aligner Solution cannot be placed definitively. This approach enables ClinCheck to eliminate inapplicable options without needing additional requests to other resource-intensive services, such as TPS, thus conserving time and resources.

After the doctor selects one or more placement options, ClinCheck updates the 3D model, illustrating placeholder objects that represent Attachment-Free Aligner Solution requests. Additionally, ClinCheck generates text comments providing detailed descriptions of these requests. With enhancements to the “ClinCheck Modifications” (CCMOD) protocol, it now facilitates the storage, retrieval, and transfer of data regarding placed Attachment-Free Activation Solution requests between various services (TPS, TPForge) and components (3D-Scene, CCBE). These modifications are saved via the TPForge service, utilizing the existing flow for saving modifications.

Placeholder objects within ClinCheck are interactive, allowing users to highlight them by clicking or tapping. Subsequently, a menu displaying possible placement options reappears, enabling users to modify previously selected options. ClinCheck incorporates and expands its attachments user experience (UX) approach to accommodate Attachment-Free Aligner Solution. Consequently, Attachment-Free Aligner Solution. Requests can also be removed by dragging and dropping them onto the Trash icon within the “Features” panel or by clicking the Trash icon when an Attachment-Free Aligner Solution placeholder object is highlighted.

At this stage, the placeholders signify the doctor's intention to incorporate Attachment-Free Aligner Solution into the treatment plan. The specific positioning of the Attachment-Free Aligner Solution is only finalized during the treatment recalculation process. This automated recalculation process within ClinCheck is referred to as Live Update. During the Live Update process, ClinCheck uses an extended version of the “ClinCheck Modifications” (CCMOD) protocol to transmit information about requested Attachment-Free Aligner Solution to the TPS.

Once the automatic treatment plan recalculation is finalized, the new treatment plan is presented within the ClinCheck UI. The potential outcomes of the Attachment-Free Aligner Solution placement requests depend on various clinical and fabrication conditions: either all requested Attachment-Free Aligner Solution were successfully placed, only some were placed, or none were placed at all. Those Attachment-Free Aligner Solution that have been placed are depicted as attachments on the lateral surfaces of the teeth and are accompanied by corresponding tooltips. Feedback regarding the outcome of Attachment-Free Aligner Solution placement is also placed within the “Align comments” section of the interface.

White Attachment is a part of the scan corresponding to the attachment once it has already been physically placed on the tooth and became its part during previous stages of treatment. When additional treatment plan is needed the white attachments may appear on new scans if they were not removed at this stage of treatment. Before invention doctors could place new attachments on teeth with white attachments during modifications of new additional plans. There was a collision for CAD-designers between old white attachment and new red one. More communication was needed to proceed with correct treatments, and it increased the time to create new treatment plan. With an invention the time for creating new treatment plans was reduced due to detecting teeth with possible collisions and offering new options for doctors. Doctors can remove all new attachments and proceed with Live update functionality, or they can submit case with collisions and CAD designers will remove white attachments in this case. There is no more misunderstanding about teeth with collisions, and it significantly reduced time for creating new treatments plan and for approvals.

New UI/UX components in ClinCheck allow users to clearly see the exact position of white attachments and collisions with new features applied on tooth. Improved UI/UX components in ClinCheck prevent collisions between modifications made by doctors and decisions made by CAD-designers. ClinCheck CCBE component detects teeth with white attachments and allow other components to interact with such teeth. Reduces time to deliver treatment to patient by providing clearer instructions about attachment placements. Helps to avoid misunderstanding and human factor between doctor and technician. Reduces number of unnecessary attachment placements on teeth with white attachments. Reduces number of iterations needed to produce approvable treatment plan.

Method for preventing inconsistency in white attachment placement in secondary treatment plans is now accessible within the ClinCheck application. This feature is available for all doctors. A general flow diagram is provided at the conclusion of this section.

During planning stage doctor can place attachment as separate virtual object on scene. During modifications process such attachments have red color and can be moved or removed manually by doctor. However, after planning process was finished and treatment has started, such attachments are placed physically on the tooth and become an inseparable part of the tooth and called “white attachment”.

Doctor can ask to plan and prepare additional treatment at the last stages of the current treatment, when attachments are still placed physically on teeth. With a new scan such attachments will appear on scene as part of tooth with white color. Such attachments can be removed for new additional treatment, or they may remain on tooth for further treatment.

Doctors may change the proposed treatment by adding new features to the treatment. Doctor must navigate to the “3D Modification Mode” within the ClinCheck application to make new changes and access the “Features” panel. Combination of available attachments will be presented on the Feature panel.

Doctor need to choose attachment from panel and place it on tooth. In case of tooth with white attachments the placeable attachment will have indication message “This tooth is marked for attachment restriction” and restriction icon below the current cursor position. The ClinCheck 3D Scene backend component (CCBE) provides data regarding the possibility of having collision on the teeth during first render of treatment. This approach allows ClinCheck to show all collision risks at the same time while doctor is trying to place new attachment. It helps to prevent new requests for each placing of attachment.

Despite the restriction message and icon, the doctor can place attachment on chosen tooth. The new attachment will turn bright red to indicate that it was placed on tooth with white attachment.

After all changes were done, doctor can choose 2 options to apply changes-click “Live Update” button to auto generate new treatment plan or click “Submit” button to send changes to CAD-designer on review.

In case of “Live update” option the warning message will appear to suggest 2 options—to remove all placed attachments that were placed on teeth with white attachments or to cancel Live Update and proceed with Submit flow. By closing this message doctor can proceed with 3D-modifications or send all current changes to CAD designers by clicking “Submit” button.

By clicking “Remove” button all new attachments with collisions will be removed form teeth with white attachments and message “Features with collision risk are removed” will appear below 3D Model. In that case all white attachment remain on teeth. After all collisions have been eliminated the doctor can proceed with Live update.

If the doctor decided to proceed with new attachments on teeth with white attachments by clicking “Submit” button, CAD designers receive treatment with two attachments on one tooth-new attachment that was placed by doctor manually and white attachment and is already placed physically on tooth. With the method for preventing inconsistency in white attachment placement in secondary treatment plans changes, CAD designers will clearly understand that in that case they need to remove white attachment from chosen tooth and keep only new attachment. Such approach significantly reduce time to prepare new treatment plan to provide it for doctor on review and increases chances of immediate approval.

Incisor/Canines Bite Ramps (hereinafter referred as I/C-BR) are modifications to the aligner shape intended to create interference with the opposing jaw. They are utilized in the treatment of deep bites as a component of Align's Deep Bite Solution. The designated placement for these Bite Ramps is solely on the lateral surface of the upper incisors, upper canines, or both.

Previously, despite I/C-BR are well known part of treatment, doctors had to request them through free-form text comments to the technician. Subsequently, they waited for a new treatment plan. To achieve desired outcomes, doctors needed to clearly specify the type and placement of I/C-BRs. Moreover, they had no feedback regarding potential placement variations or feasibility before the technician's response.

Now, with the advancement of automated treatment planning software (TPS), it's possible to incorporate I/C-BRs into the treatment plan without technician involvement. This enhancement built upon the existing mechanism known as ClinCheck Live Update, incorporating data regarding requested Bite Ramps and delivering feedback on the outcomes.

This innovation introduces a new workflow for I/C-BR placement, encompassing new UI/UX elements for requesting and displaying feedback, alongside changes in data flow to accommodate TPS recalculation capabilities.

Upgraded TPS now provides a recalculated treatment plan with Incisor/Canines Bite Ramps (hereinafter referred as I/C-BR) by utilizing the existing codebase shared with Treat for automated treatment plan recalculation. Extended data flow between TPS and ClinCheck allows the transfer of I/C-BR request and result data. ClinCheck CCBE component adopts logic which allows to provide immediate feedback about I/C-BR placement possibilities. New UI/UX components in ClinCheck enable interactive placement of I/C-BR requests and visually display where and how they can be placed. Improved UI/UX components in ClinCheck highlight the outcome of I/C-BR requests effectively.

Reduces time to deliver treatment with applied bite ramps to patient. Helps to avoid misunderstanding and human factor between doctor and technician. Reduces number of iterations needed to produce approvable treatment plan.

Automated placement of Incisor/Canine Bite Ramps is now accessible within the ClinCheck application. A general flow diagram is provided at the conclusion of this section. To initiate the Bite Ramps placement process, the doctor must navigate to the “3D Modification Mode” within the ClinCheck application and access the Features panel. If available, the “Bite Ramps” tool button will be located there.

There are two methods for the doctor to activate the “Bite Ramps” tool in the “Features panel”: Click the “Bite Ramps” tool button to toggle it into an active state. Then, move the cursor to the desired location on the 3D model and click again. Alternatively, the doctor can drag and drop the “Bite Ramps” tool directly onto the desired location on the 3D model.

While dragging or hovering over the “Bite Ramps” tool, ClinCheck provides the doctor with feedback regarding whether the Bite Ramps can be placed at the current position. If Bite Ramps cannot be placed at the current position, a red cross will appear along with the cursor. Once the doctor drops the “Bite Ramps” tool to the desired location or clicks on the desired location when the “Bite Ramps” tool is active, a menu with possible placement options will appear.

The ClinCheck 3D Scene backend component (CCBE) supplies data regarding the possibility of placing Bite Ramps on the teeth below the current cursor position. It employs a first-approach logic, determining where and which Bite Ramps cannot be placed definitively. This approach enables ClinCheck to eliminate inapplicable options without needing additional requests to other resource-intensive services, such as TPS, thus conserving time and resources.

After the doctor selects one or more placement options, ClinCheck updates the 3D model, illustrating placeholder objects that represent Bite Ramps requests. Additionally, ClinCheck generates text comments providing detailed descriptions of these requests. Modified “ClinCheck Modifications” (CCMOD) protocol now enables the storage, retrieval, and transfer of data regarding placed Bite Ramps requests between various services (TPS, TPForge) and components (3D-Scene, CCBE). These modifications are saved via the TPForge service, utilizing the existing flow for saving modifications.

Placeholder objects within ClinCheck are interactive, allowing users to highlight them by clicking or tapping. Subsequently, a menu displaying possible placement options reappears, allowing users to modify previously selected options. ClinCheck incorporates and expands its existing user experience (UX) approach of working with attachments to accommodate Bite Ramps related modifications. Consequently, Bite Ramps requests can also be removed by dragging and dropping them onto the Trash icon within the “Features” panel or by clicking the Trash icon when a Bite Ramps placeholder object is highlighted.

At this stage, the placeholders signify the doctor's intention to incorporate Bite Ramps into the treatment plan. The specific form, type, and positioning of the Bite Ramps are only finalized during the treatment recalculation process. This automated recalculation process within ClinCheck is referred to as Live Update. During the Live Update process, ClinCheck uses an extended version of the “ClinCheck Modifications” (CCMOD) protocol to transmit information about requested Bite Ramps to the TPS.

Once the automatic treatment plan recalculation is finalized, the new treatment plan is presented within the ClinCheck UI. The potential outcomes of the Bite Ramps placement requests depend on various clinical and fabrication conditions: either all requested Bite Ramps were successfully placed, only some were placed, or none were placed at all. Those Bite Ramps that have been placed are depicted as attachments on the lateral surfaces of the teeth and are accompanied by corresponding tooltips. Report regarding the outcome of Bite Ramps placement is also placed within the “Align comments” section of the interface.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for designing a dental treatment plan, the system comprising:
a user interface configured to display graphical data and receive user input;
one or more processors; and
a memory coupled to the one or more processors, the memory configured to store computer-program instructions that, when executed by the one or more processors cause the system to implement a method comprising:
displaying, on a user interface, a three-dimensional (3D) digital model of a dentition based on a dental treatment plan, wherein the 3D digital model includes a surface of at least one tooth with a first attachment;
receiving, through the user interface, a user input to locate a second attachment on the surface of the at least one tooth;
determining, by the one or more processors, an interference between the first attachment and the second attachment;
generating an updated dental treatment plan based on the interference between the first attachment and the second attachment; and
displaying an updated 3D digital model based on the updated dental treatment plan.

2. The system of claim 1, wherein determining the interference comprises executing a geometric intersection algorithm configured to detect volumetric overlap between 3D mesh regions defining the first and second attachment surfaces.

3. The system of claim 1, wherein the updated dental treatment plan removes the first attachment.

4. The system of claim 1, wherein the updated dental treatment plan uses the first attachment in place of the second attachment.

5. The system of claim 1, further comprising displaying, on the user interface, the interference between the first attachment and the second attachment.

6. The system of claim 1, wherein generating the updated dental treatment plan comprises:
sending the user input to a remote computing device configured to generate the updated dental treatment plan based on the dental treatment plan; and
returning the updated dental treatment plan to the user interface.

7. The system of claim 1, wherein generating the updated dental treatment plan comprises:
sending the user input to a designer to generate the updated dental treatment plan based on the dental treatment plan; and
returning the updated dental treatment plan to the user interface.

8. The system of claim 1, further comprising:
displaying, on the user interface, the interference between the first attachment and the second attachment, based on determining the interference between the first attachment and the second attachment.

9. The system of claim 8, further comprising:
displaying, on the user interface, a user message to select either the first attachment or the second attachment; and
updating the dental treatment plan based on the user selection.

10. A non-transitory computer-readable storage medium comprising instructions that, when executed by one or more processors of a device, cause the device to perform operations comprising:
displaying, on a user interface, a three-dimensional (3D) digital model of a dentition based on a dental treatment plan, wherein the 3D digital model includes a surface of at least one tooth with a first attachment;
receiving, through the user interface, a user input to locate a second attachment on the surface of the at least one tooth;
determining, by the one or more processors, an interference between the first attachment and the second attachment;
generating an updated dental treatment plan based on the interference between the first attachment and the second attachment; and
displaying an updated 3D digital model based on the updated dental treatment plan.

11. A system for designing a dental treatment plan, the system comprising:
a user interface configured to display graphical data and receive user input;
one or more processors; and
a memory coupled to the one or more processors, the memory configured to store computer-program instructions that, when executed by the one or more processors cause the system to implement a method comprising:
displaying, on a user interface, a three-dimensional (3D) digital model of a dentition based on a patient's dental treatment plan;
receiving, through the user interface, a user input selecting a location for an occlusal feature on a tooth of the 3D digital model;
determining, by the one or more processors, whether the occlusal feature can be implemented at the selected location;
generating an updated dental treatment plan based at the selected location of the occlusal feature; and
displaying an updated 3D digital model based on the updated dental treatment plan.

12. The system of claim 11, wherein determining whether the occlusal feature can be implemented at the selected location comprises applying a plurality of rule-based constraints.

13. The system of claim 11, wherein the occlusal feature comprises a bite ramp or a disocclusion feature.

14. The system of claim 11, wherein the occlusal feature is integral to a dental appliance manufactured in accordance with the updated dental treatment plan.

15. The system of claim 11, further comprising: displaying, on the user interface, an indication to a user whether the occlusal feature can be implemented at the selected location.

16. The system of claim 11, further comprising receiving through the user interface the user input to remove the occlusal feature.

17. The system of claim 11 further comprising:
sending the user input selecting the location for an occlusal feature to a remote computing device configured to generate the updated dental treatment plan; and
returning the updated dental treatment plan that includes the occlusal feature to the user interface.

18. The system of claim 11, further comprising receiving, through the user interface, a modification of a previously selected location of the occlusal feature.

19. The system of claim 11, determining whether the occlusal feature can be implemented at the selected location includes determining whether the occlusal feature can be implemented with respect to a tooth associated with the selected location.

20. The system of claim 11, wherein generating the updated dental treatment plan comprises:
sending the user input to a remote computing device configured to generate the updated dental treatment plan based on the user input; and
returning the updated dental treatment plan to the user interface.
